(19) United States
(12) Patent Application Publication
(10) Pub. No.: US 2009/0306083 A1
(43) Pub. Date: Dec. 10, 2009

(54) Use of Trifluoromethyl Substituted Benzamides in teh Treatment of Neurological Disorders

(75) Inventors: Rajeev Sivasankaran, Acton, MA (US); Kaspar Zimmermann, Oberwil (CH)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/373,592

(22) PCT Filed: Jul. 11, 2007
(86) PCT No.: PCT/US2007/073238

§ 371 Date: Jan. 13, 2009

Related U.S. Application Data
(60) Provisional application No. 60/807,210, filed on Jul. 13, 2006.

(51) Int. Cl. A61K 31/50; A61K 31/517; A61K 31/47; A61K 31/428

(52) U.S. Cl. 514/248; 514/266.1; 514/311; 514/367

(57) ABSTRACT

The invention relates to methods of using the compounds of the invention, including trifluoromethyl substituted benzamide compounds and salts thereof, as well as pharmaceutical compositions comprising the same, in the treatment of Eph receptor-related (e.g., neurological) injuries and disorders. The invention also relates to modulating the activity of an Eph receptor in a cell, stimulating neural regeneration, and reversing neuronal degeneration, by administering a compound of the invention to a cell or subject in an effective amount.

### BACKGROUND OF THE INVENTION

Injury to the adult mammalian central nervous system (CNS) is often characterized by axonal impairment, including an inability of severed axons to regrow to their targets, resulting in permanent paralysis in subjects with said injuries. There currently exists no cure for patients who have suffered such CNS-related trauma as spinal cord injury (SCI), which is very often accompanied by debilitating clinical conditions like paraplegia or quadriplegia.

Axonal regeneration (e.g., post-injury) is prevented by a host of inhibitory influences in the adult CNS, among them inhibitory myelin proteins and the formation of a glial scar. Considerable progress has been made in identifying molecules associated with myelin inhibition (e.g., Nogo, myelin-associated glycoprotein (MAG), and oligodendrocyte-myelin glycoprotein (OMgp)), but relatively little is known about the glial scar which is formed as a response of glial cells to injury. (GrandPre, T., et al. (2000) Nature, 403(6768): 439; Fournier, A. E. et al. (2001) Nature 409(6818): 341; Wang, K. C., et al. (2002) Nature 417(6892): 941; and Wang, K. C., et al. (2002) Nature 420(6911): 74). Glial scarring is characterized by astrocytic gliosis, in which normally quiescent astrocytes proliferate and grow hypertrophic in response to injury, and otherwise form a physical and chemical barrier to axon regeneration. (Silver, J., et al. (2004) Nat Rev Neurosci 5(2): 146; and Morgenstern, D. A., et al. (2002) Prog Brain Res. 137: 313). Although a range of glial cells contribute to scar formation, the astrocytic response (i.e., astrocytic gliosis) is thought to be the primary mechanism for this occurrence.

The Eph receptor tyrosine kinase subfamily is thought to be the largest subfamily of transmembrane receptor tyrosine kinases, and with its ligands, the ephrins, is responsible for governing proper cell migration and positioning during neural development, presumably through modulating intercellular repulsion. (Pasquale, E. (1997) Curr. Opin. Cell Biol. 9:608-615) (Orioli and Klein (1997) Trends in Genetics 13:354-359). Eph receptors are closely related, and actively signal when bound to their ephrin ligands (their effects are mediated by cell-to-cell contacts), with which they are capable of both forward and bi-directional signaling. (Murai, K. K., et al. (2003) J Cell Sci. 116: 2823).

The Eph receptors are known regulators of neural development, with roles in the regulation of migrating cells or axons, the establishment of tissue patterns and topographic maps in distinct regions of the developing brain, and the regulation of synapse formation and plasticity. Eph receptors, including EphA4 and EphA7, are upregulated after spinal cord damage or deafferentation. (Miranda, et al. (1999) Exp Neurol 156:218; Willson, et al. (2002) Cell Transplantation 11:229); therefore, their inhibition is viewed as a potential therapeutic strategy for the treatment of neurological disorders.

A significant step toward curing or ameliorating complications resulting from spinal cord injury has been wanting, owing to the complex and multi-factorial nature of SCIs. In vivo studies have been performed to assess recovery following SCI by blocking either myelin inhibitors (GrandPre, T., et al. (2002) Nature 417(6888): 547; Kim, J. E., et al. (2003) Neuron 38(2): 187), chondroitin sulfate proteoglycans (Bradbury, E. J., et al. (2002) Nature 416 (6881): 636) or signaling molecules downstream of both of these (Fournier, A. E., et al. (2003) J Neurosci. 23(4): 1416; and Sivasankaran, R., et al. (2004) Nat Neurosci. 7(3): 261), with only marginal success. Experimental inhibition of Eph receptors, however, has revealed considerable axon regeneration following injury and suppressed astrocytic gliosis, leading to a dramatic reduction in glial scarring, and making these receptors an ideal therapeutic target for spinal cord injury and stroke, which also results in axonal damage and gliosis. Strategies and therapeutics designed to block Eph receptor function therefore herald a significant advance in the treatment of CNS-related disorders, and could presumably lead to vastly improved recovery following neural injury such as SCI, stroke and other neurodegenerative disorders.

### SUMMARY OF THE INVENTION

The invention relates to methods of using of the compounds of the invention for the treatment of Eph receptor-related (e.g., neurological) injuries and disorders, and methods of using pharmaceutical preparations comprising the compounds of the invention in the treatment of Eph receptor-related (e.g., neurological) injuries and disorders.

The invention also relates to methods of modulating the activity of an Eph receptor in a cell by contacting the cell with an effective amount of the compounds of the invention. In certain embodiments, Eph receptors can be modulated either in vitro or in vivo.

The invention also relates to methods of stimulating and promoting neural regeneration (such as axon regeneration following spinal cord injury), and reversing neuronal degeneration due to traumatic injury, hypoxic conditions, or infarct (e.g., as in stroke or nerve degeneration that is an underlying cause in multiple sclerosis and other neurodegenerative diseases). One way in which this can be achieved is through the administration to a mammal of a compound of the invention in an amount that is sufficient to stimulate and promote neural regeneration (such as axon regeneration) or reverse neuronal degeneration. The compounds of the invention can be delivered to both normal and injured cells. In some embodiments, the compounds of the invention inhibit the phosphorylation of an Eph receptor. In other embodiments, the compounds of the invention inhibit the binding of ephrin ligands to Eph receptors.

The invention also relates to methods for delivering a therapeutic agent to a cell, such as via a conjugate which comprises a therapeutic agent (e.g., a linking reagent) linked to compound of the invention.

As described herein and in PCT publication WO06/015859 (the contents of which are hereby incorporated by reference), the compounds of the invention, e.g., trifluoromethyl substituted benzamide compounds, are, among other things, useful as protein kinase inhibitors and thus in the treatment of protein kinase-related disorders. By way of example, the compounds of the invention are useful as receptor tyrosine kinase inhibitors, such as Ephrin receptor kinase inhibitors, and can therefore be used to treat, e.g., neurological injuries and disorders.

### BRIEF DESCRIPTION OF THE FIGURE(S)

FIG. 1 shows inhibition of EphA4 ligand-dependent phosphorylation (from samples subjected to EphA4 immunoprecipitation followed by a phosphotyrosine Western blot). FIG. 1, lanes 1 and 2 show EphA4 phosphorylation in control (untreated) and ephrinB3-Fc stimulated cells following serum starvation. All other lanes represent samples from cells stimulated with ephrinB3-Fc in the presence of varying concentrations (as indicated) of tested Eph inhibitors.

FIG. 2 shows a graphical representation of experimentally-determined neurite outgrowth inhibition. The Y axis shows average neurite length in microns. The bars represent, from left to right, myelin, PDL, and tested compounds of the invention.

FIGS. 3A and 3B show a picture and graphical representation, respectively, of the experimental determination that Eph receptor inhibitors block astrocyte migration induced by cytokines (TGF-α, LIF, and IFN). FIG. 3A, left to right, represents serum free, cytokines, and cytokines plus Compound 3, respectively. In FIG. 3B, the Y axis shows relative area of cell migration compared to control (serum free=1). From left to right, the bars represent serum free, cytokines, and cytokine plus 10 nM Compound 3.

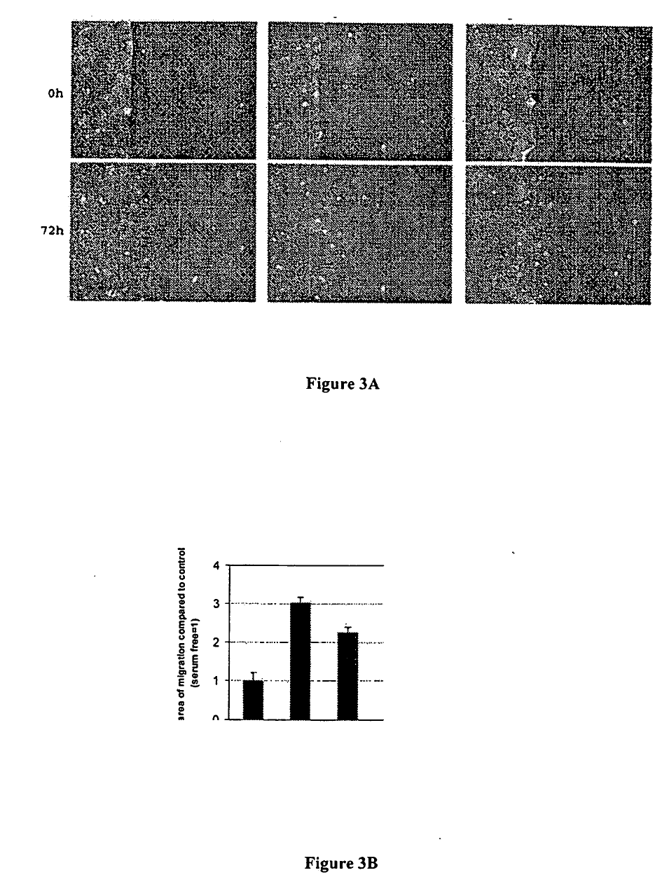

FIG. 4 demonstrates that Eph receptor inhibitors block EphA4 phosphorylation in vivo in mouse brain (brain homogenate lysates were subjected to EphA4 immunoprecipitation followed by a phospho-tyrosine Western blot). Animals were given an i.v dose of relevant compound and sacrificed 25 minutes or 1 hour after dosing (0.25 h or 1 h), the brains were removed and subjected to EphA4 immunoprecipitation followed by a phospho-tyrosine Western blot. Four animals were used as controls and three animals each were used per time point for each drug. Compound 3 is shown second from the top.

### DETAILED DESCRIPTION OF THE INVENTION

The invention in particular relates to trifluoromethyl substituted benzamide compounds of the formula I,

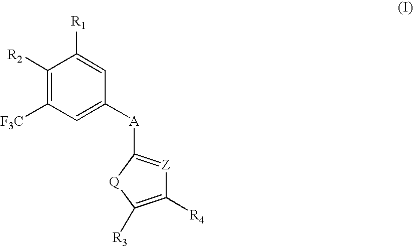

wherein

R¹ is hydrogen or —N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;

R₂ is hydrogen or —CH₂—N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;

with the proviso, that at least one of R₁ and R₂ is hydrogen;
R₃ is halo or C₁-C₇-alkyl;

R₄ is bicyclic heterocyclyl selected from the group consisting of

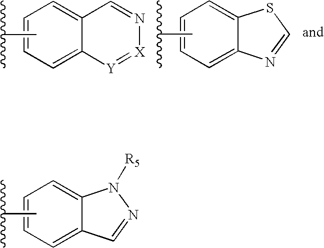

wherein

X is CH, N or C—NH₂;

Y is CH or N;

with the proviso that not both of X and Y are N simultaneously;

and R₅ is hydrogen, C₁-C₇-alkyl or unsubstituted or substituted phenyl;

A is —C(=O)—NH— (with the —NH— bound to the ring comprising Q and Z in formula I) or —NH—C(═O)— (with the —C(═O)— bound to the ring comprising Q and Z in formula I);

Z is CH or N; and

Q is —S— or —CH═CH—;

or a (preferably pharmaceutically acceptable) salt thereof where one or more salt-forming groups are present.

In a preferred embodiment of the invention, the compounds of the invention are selected from the group consisting of N-(3-isoquinolin-7-yl-4-methyl-phenyl)-3-trifluoromethyl-benzamide (“Compound 1”), N-(4-methyl-3-quinazolin-6-yl-phenyl)-3-trifluoromethyl-benzamide (“Compound 2”), 3-isoquinolin-7-yl-4-methyl-N-(3-trifluoromethyl-phenyl)-benzamide (“Compound 3”), 4-methyl-3-quinazolin-6-yl-N-(3-trifluoromethyl-phenyl)-benzamide (“Compound 4”), N-(3-benzothiazol-6-yl-4-methyl-phenyl)-3-trifluoromethyl-benzamide (“Compound 5”), 3-benzothiazol-6-yl-4-methyl-N-(3-trifluoromethyl-phenyl)-benzamide (“Compound 6”), N-(4-methyl-3-phthalazin-6-yl-phenyl)-3-trifluoromethyl-benzamide (“Compound 7”), 4-methyl-3-phthalazin-6-yl-N-(3-trifluoromethyl-phenyl)-benzamide (“Compound 8”), N-(3-benzothiazol-5-yl-4-methyl-phenyl)-3-trifluoromethyl-benzamide (“Compound 9”), and 3-benzothiazol-5-yl-4-methyl-N-(3-trifluoromethylphenyl)benzamide (“Compound 10”).

In an embodiment of the invention, the compounds of the invention are used in methods of treatment of Eph receptor-related (e.g., neurological) injuries and disorders.

In another embodiment of the invention, pharmaceutical compositions prepared from the compounds of the invention are used in methods of treatment of Eph receptor-related (e.g., neurological) injuries and disorders. The pharmaceutical compositions preferably comprise a compound of the invention an acceptable pharmaceutical carrier. Carriers are described in greater detail herein.

In another embodiment of the invention, the compounds of the invention are used to contact a cell, in order to modulate the activity of an Eph receptor therein. The cell can be contacted in vitro or in vivo, in an effective amount of the compounds of the invention to modulate Eph receptors therein.

In yet another embodiment of the invention, the compounds of the invention are used in methods of stimulating and promoting neural regeneration (such as axon regeneration), and reversing neuronal degeneration due to traumatic injury, stroke, multiple sclerosis and neurodegenerative diseases. One way in which this can be achieved is through the administration to a mammal of a compound of the invention in an amount that is sufficient to stimulate and promote neural regeneration (such as axon regeneration) or reverse neuronal degeneration. The compounds of the invention can be delivered to both normal and injured cells. In some embodiments, the compounds of the invention inhibit the phosphorylation of an Eph receptor. In other embodiments, the compounds of the invention inhibit the binding of ephrin ligands to Eph receptors.

In still yet another embodiment of the invention, the compounds of the invention are used in methods for delivering a therapeutic agent to a cell, such as via a conjugate comprising said therapeutic agent linked to compound of the invention. As described in greater detail herein, the therapeutic agent can be a linking reagent.

### DEFINITIONS

The general terms or symbols used hereinbefore and hereinafter preferably have within, the context of this disclosure, the following meanings, unless otherwise indicated:

As used herein, “Eph receptor” means a receptor tyrosine kinase that belongs to the Eph family, including EphA2, EphA4, EphA5, EphA7, EphB2 and EphB4. This family is reviewed, for instance, in Pasquale, E. (1997) Curr. Opin. Cell Biol. 9:608-615; and Orioli and Klein (1997) Trends in Genetics 13:354-359.

“Eph receptor-related injuries and disorders” include neurological injuries and disorders, including but not limited to spinal cord injury (SCI); quadriplegia, hemiplegia, and paraplegia, including injury-caused and hereditary forms; neuropathies; and CNS related disorders (e.g., bacterial and viral meningitis).

“Eph-receptor-related injuries and disorders” also includes neuronal degeneration resulting from hypoxic conditions, or from an infarct as in stroke. This condition can result in deficits in motor, sensory and cognitive functions, in large part due to the inability of injured axons to regenerate and undergo synaptic reorganization. As with SCI, stroke is followed by the formation of a glial scar at the site of infarction, and inhibiting EphA4 (e.g., as with the compounds of the invention) can inhibit scarring and thus enable improved regeneration and reorganization of connections. An in vitro model of stroke using astrocyte—hippocampal neuron co-cultures, has been shown that the inter-astrocytic gap-junctions are very important for the survival of neurons following hypoxic stress. (Blanc, E. M., et al. (1998) J Neurochem, 70(3): 958). As Eph receptors are known to be involved in signaling at gap-junctions, this represents another potential implication of EphA4 in ischemic stroke (Mellitzer, G., et al. (1999) Nature, 400(6739): 77).

As used herein, the term “treatment” includes both prophylactic or preventive treatment as well as curative or disease suppressive treatment, including treatment of patients at risk of neurological disorders, as well as ill and injured patients. This term further includes the treatment for the delay of progression of the disease.

The following abbreviations are used herein to represent commonly used terms (in parenthesis) in the present application, including but not limited to the examples section: DMSO (dimethylsulfoxide); ES-MS (electrospray mass spectrometry); EtOAc (Ethyl Acetate); HPLC (high-pressure liquid chromatography); mL (mililiter(s)); NMR (nuclear magnetic resonance); RT (room temperature); ^{A}t_{RET} (HPLC retention time in minutes (method A)); ^{B}t_{RET} (HPLC retention time in minutes (method B)); ^{C}t_{RET} (HPLC retention time in minutes (method C)); ^{D}t_{RET} (HPLC retention time in minutes (method D)); TFA (trifluoroacetic acid); THF (tetrahydrofuran); TMSCI (Trimethylsilyl chloride).

In each case where a waved line vertical to a bond is used, this marks the bond where a given moiety is bound to the rest of the corresponding molecule.

The term “lower” or “C₁-C₇—” defines a moiety with up to and including maximally 7, especially up to and including maximally 4, carbon atoms, said moiety being branched or straight-chained. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Unsubstituted or substituted phenyl is unsubstituted or substituted by one or more, preferably one or two substituents, wherein the substituents are independently selected from any one or more of the functional groups including: halo, lower alkyl, substituted lower alkyl, such as halo lower alkyl e.g. trifluoromethyl, lower alkenyl, lower alkynyl, lower alkanoyl, lower alkoxy, hydroxy, etherified or esterified hydroxy, amino, mono- or disubstituted amino, such as mono- or di-lower alkylamino, amino lower alkoxy; lower alkanoylamino; amidino, nitro, cyano, cyano-lower alkyl, carboxy, esterified carboxy, especially lower alkoxy carbonyl, e.g. methoxy carbonyl, n-propoxy carbonyl or iso-propoxy carbonyl, lower alkanoyl, benzoyl, carbamoyl, N-mono- or N,N-disubstituted carbamoyl, such as N-mono- or N,N-di-lower alkylcarbamoyl or N-mono- or N,N-di-(hydroxy-lower alkyl)-carbamoyl, amidino, guanidino, ureido, mercapto, sulfo, lower alkylthio, sulfonamido, benzosulfonamido, sulfono, phenyl, phenyl-lower alkyl, such as benzyl, phenoxy, phenyl-lower alkoxy, such as benzyloxy, phenylthio, phenyl-lower alkylthio, lower alkyl-phenylthio, lower alkylsulfinyl, phenylsulfinyl, phenyl-lower alkylsulfinyl, alkylphenylsulfinyl, lower alkanesulfonyl, phenylsulfonyl, phenyl-lower alkylsulfonyl, alkylphenylsulfonyl, halogen-lower alkylmercapto, halogen-lower alkylsulfonyl, such as trifluoromethane sulfonyl, dihydroxybora (—B(OH)₂), lower alkylene dioxy bound at adjacent C-atoms of the ring, such as methylene dioxy, phosphono (—P(═O)(OH)₂), hydroxy-lower alkoxy phosphoryl or di-lower alkoxy-phosphoryl, or —NRₐR_{b}, wherein Rₐ and R_{b} can be the same or different and are independently H; lower alkyl (e.g. methyl, ethyl or propyl); or Rₐ and R_{b} together with the N atom form a 3- to 8-membered heterocyclic ring containing 1-4 nitrogen, oxygen or sulfur atoms (e.g. piperazinyl, lower alkyl-piperazinyl, azetidinyl, pyrrolidinyl, piperidino, morpholinyl, imidazolinyl).

As used herein, “compounds of the invention” include, compounds of formula (I), including trifluoromethyl substituted benzamides. Compounds of the invention also refers to those compounds referred to herein as “Compound [number].” Further definition of numbered compounds can be found in the Examples section of the present application.

“Aryl” is an aromatic radical having 6 to 14 carbon atoms, especially phenyl, naphthyl, indenyl, azulenyl, or anthryl, and is unsubstituted or substituted by one or more, preferably one or two substituents, wherein the substituents are selected from any of the functional groups defined below, and including: lower halo, alkyl, substituted alkyl, halo lower alkyl e.g. trifluoromethyl, lower alkenyl, lower alkynyl, lower alkanoyl, lower alkoxy, hydroxy, etherified or esterified hydroxy, amino, mono- or disubstituted amino, amino lower alkyl, amino lower alkoxy; acetyl amino; amidino, halogen, nitro, cyano, cyano lower alkyl, carboxy, esterified carboxy especially lower alkoxy carbonyl, e.g. methoxy carbonyl, n-propoxy carbonyl or iso-propoxy carbonyl, alkanoyl, benzoyl, carbamoyl, N-mono- or N,N-disubstituted carbamoyl, carbamates, alkyl carbamic acid esters, amidino, guanidino, urea, ureido, mercapto, sulfo, lower alkylthio, sulfoamino, sulfonamide, benzosulfonamide, sulfonate, phenyl, benzyl, phenoxy, benzyloxy, phenylthio, phenyl-lower alkylthio, alkylphenylthio, lower alkylsulfinyl, phenylsulfinyl, phenyl-lower alkylsulfinyl, alkylphenylsulfinyl, lower alkanesulfonyl, phenylsulfonyl, phenyl-lower alkylsulfonyl, alkylphenylsulfonyl, halogen-lower alkylmercapto, halogen-lower alkylsulfonyl, such as especially trifluoromethane sulfonyl, dihydroxybora (—B(OH)₂), heterocyclyl, and lower alkylene dioxy bound at adjacent C-atoms of the ring, such as methylene dioxy, phosphono (—P(═O)(OH)₂), hydroxy-lower alkoxy phosphoryl or di-lower alkoxyphosphoryl, carbamoyl, mono- or di-lower alkylcarbamoyl, mono- or di-(hydroxy-lower alkyl)-carbamoyl, or —NR₄R₅, wherein R₄ and R₅ can be the same or different and are independently H; lower alkyl (e.g. methyl, ethyl or propyl); or R₄ and R₅ together with the N atom form a 3- to 8-membered heterocyclic ring containing 1-4 nitrogen, oxygen or sulfur atoms (e.g. piperazinyl, lower alkyl-piperazinyl, azetidinyl, pyrrolidinyl, piperidino, morpholinyl, imidazolinyl).

Aryl is more preferably phenyl which is either unsubstituted or independently substituted by one or two substituents selected from a solubilizing group selected from the group consisting of: halo (such as Cl or Br); hydroxy; lower alkyl (such as C₁-C₃ lower alkyl); aryl (such as phenyl or benzyl); amino; amino lower alkyl (such as dimethylamino); acetyl amino; amino lower alkoxy (such as ethoxyamine); lower alkyl (such as methyl); alkoxy (such as methoxy or benzyloxy where the benzyl ring may be substituted or unsubstituted, such as 3, 4-dichlorobenzyloxy); sulfoamino; substituted or unsubstituted sulfonamide (such as benzo sulfonamide, chlorobenzene sulfonamide or 2,3-dichloro benzene sulfonamide); substituted or unsubstituted sulfonate (such as chloro-phenyl sulfonate); substituted urea (such as 3-trifluoro-methyl-phenyl urea or 4-morpholin-4-yl-3-triflurormethyl-phenyl-urea); alkyl carbamic acid ester or carbamates (such as ethyl-N-phenyl-carbamate) or —NR₄R₅, wherein R₄ and R₅ can be the same or different and are independently H; lower alkyl (e.g. methyl, ethyl or propyl); or R₄ and R₅ together with the N atom form a 3- to 8-membered heterocyclic ring containing 1-4 nitrogen, oxygen or sulfur atoms (e.g. piperazinyl, lower alkyl-piperazinyl, pyridyl, indolyl, thiophenyl, thiazolyl, morpholinyl n-methyl piperazinyl, benzothiophenyl, azetidinyl, pyrrolidinyl, piperidino or imidazolinyl);

A heteroaryl group is preferably monocyclic, but may be bi- or tri-cyclic, and comprises 3-24, preferably 4-16 ring atoms, wherein at least one or more, preferably one to four ring carbons are replaced by a heteroatom selected from O, N or S. Preferably the heteroaryl group is selected from pyridyl, indolyl, pyrimidyl, pyrazolyl, oxazolyl, thiophenyl, benzothiophenyl, 2H-pyrrolyl, pyrrolyl, imidazolyl, benzimidazolyl, pyrazolyl, indazolyl, purinyl, pyrazinyl, pyridazinyl, 4H-quinolizinyl, isoquinolyl, quinolyl, phthalazinyl, naphthyridinyl, quinoxalyl, quinazolinyl, quinnolinyl, indolizinyl, 3H-indolyl, isoindolyl, isoxazolyl, thiazolyl, isothiazolyl, triazolyl, tetrazolyl, furazanyl and benzo[d]pyrazol.

More preferably the heteroaryl group is selected from the group consisting of pyridyl, indolyl, pyrimidyl, pyrazolyl, oxazolyl, thiophenyl or benzothiophenyl.

The heteroaryl group may be unsubstituted or substituted by one or more substituents selected from the group defined above as substituents for aryl, most preferably by hydroxy, halogen, lower alkyl, such as methyl or lower alkoxy, such as methoxy or ethoxy.

“Aliphatic,” as used herein, refers to any non-aromatic carbon based residue. Examples of aliphatic residues include substituted or unsubstituted alkyl, cycloalkyl, alkenyl and alkynyl.

“Alkyl” includes lower alkyl preferably alkyl with up to 7 carbon atoms, preferably from 1 to and including 5, and is linear or branched; preferably, lower alkyl is pentyl, such as n-pentyl, butyl, such as n-butyl, sec-butyl, isobutyl, tert-butyl, propyl, such as n-propyl or isopropyl, ethyl or methyl. Preferably lower alkyl is methyl, propyl or tert-butyl.

A cycloalkyl group is preferably cyclopentyl, cyclohexyl or cycloheptyl, and may be unsubstituted or substituted by one or more, especially one or two, substituents selected from the group defined above as substituents for aryl, most preferably by lower alkyl, such as methyl, lower alkoxy, such as methoxy or ethoxy, or hydroxy.

Alkenyl and alkynyl preferably have up to 7 carbon atoms, preferably from 1 to and including 5, and can be linear or branched.

Alkyl, cycloalkyl, alkenyl and alkynyl can be substituted or unsubstituted, and when substituted may be with up to 3 substituents including other alkyl, cycloalkyl, alkenyl, alkynyl, any of the substituents defined above for aryl or any of the functional groups defined below.

“Halo” or “halogen” is preferably fluoro, chloro, bromo or iodo, most preferably fluoro, chloro or bromo.

The term “connecting atom or group” as used herein includes alkyl, (such as —CH₂—); oxy —O—; keto —CO—; thio —S—; sulfonyl —SO₂—; sulfoxides —SO—; amines —NH— or —NR—; carboxylic acid; alcohol; esters (—COO—); amides (—CONR—, —CONHR′—); sulfonamides (, —SO₂NH—, —SO₂NR′—); sulfones (—SO₂—); sulfoxides (—SO—); amino-group; ureas ( —NH—CO—NH—, —NR—CO—NH—, —NH—CO—NR—, —NR—CO—NR—); ethers (—O—); carbamates (—NH—CO—O—, —NR—CO—O—); or inverse amides sulfonamides and esters (—NH—CO—, —NR—CO—, —NH—SO₂—, —NR—SO₂—, —OOC—).

The term “functional group” as used herein includes: carboxylic acid; hydroxyl; halogen; cyano (—CN); ethers (—OR); ketones (—CO—R); esters (—COOR); amides (—CONH2, —CONHR, —CONRR′); thioethers (—SR); sulfonamides (—SO₂NH₂, —SO₂NHR, —SO₂NRR′); sulfones (—SO₂—R); sulfoxides (—SO—R); amines (—NHR, NR′R); ureas (—NH—CO—NH₂, —NH—CO—NHR); ethers (—O—R); halogens; carbamates (—NH—CO—OR); aldehyde-function (—CHO); then also inverse amides; sulfonamides and esters (—NH—CO—R, —NH—SO₂—R, —OOC—R);

R and R′ are the same are different and may be H or are any aliphatic, aryl or heteroaryl moiety as defined above.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous solutions, or can be isolated especially in solid form.

Such salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxy-maleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds” hereinbefore and hereinafter, especially to the compound(s) of the formula I, is to be understood as referring also to one or more salts thereof or a mixture of a free compound and one or more salts thereof, as appropriate and expedient and if not mentioned otherwise.

Where the plural form is used for compounds, salts, pharmaceutical preparations, diseases, disorders and the like, this is intended to mean also a single compound, salt, pharmaceutical preparation, disease or the like, and vice versa.

In view of the close relationship between the compounds in free form and those in the form of their salts, including those salts that can be used as intermediates, for example in the purification or identification of the compounds, tautomers or tautomeric mixtures and their salts, any reference to the compounds hereinbefore and hereinafter especially the compounds of the formula (I), is to be understood as referring also to the corresponding tautomers of these compounds, especially of compounds of the formula (I), tautomeric mixtures of these compounds, especially of compounds of the formula (I), or salts of any of these, as appropriate and expedient and if not mentioned otherwise.

Where “a compound . . . , a tautomer thereof; or a salt thereof” or the like is mentioned, this means “a compound . . . , a tautomer thereof, or a salt of the compound or the tautomer.”

Any asymmetric carbon atom may be present in the (R)-, (S)- or (R,S)-configuration, preferably in the (R)- or (S)-configuration. Substituents at a ring at atoms with saturated bonds may, if possible, be present in cis-(=Z-) or trans (=E-) form. The compounds may thus be present as mixtures of isomers or preferably as pure isomers, preferably as enantiomer-pure diastereomers or pure enantiomers.

The compounds of formula (I) have valuable pharmacological properties and are useful in the treatment of Eph receptor-related (e.g., neurological) injuries and disorders, e.g., as drugs to treat neurological diseases.

CNS-Related Injuries and Disorders

Injury to the central nervous system usually results in very limited, if any, regeneration of lesioned axons, with subsequent permanent impairment of function. Although some CNS neurons appear to lose the intrinsic ability to regenerate neurites postnatally, many others, such as corticospinal tract (CST) neurons, appear able to regenerate, but are inhibited from doing so by the environment of the injury site. (Goldberg et al., (2002) Science 296: 1860). Major impediments to CNS regeneration are the presence of myelin inhibitors and astrocytic gliosis.

Axonal regeneration is prevented by a host of inhibitory influences in the adult CNS, among them inhibitory myelin proteins and the formation of a glial scar. Although considerable progress has been made in identifying molecules associated with myelin inhibition (e.g., Nogo, myelin-associated glycoprotein (MAG), and oligodendrocyte-myelin glycoprotein (OMgp)), targeting those proteins for the treatment or amelioration of neurological disorders is an incomplete solution. Blocking individual myelin proteins or their common receptor in vivo after spinal cord injury can result in partial axon regeneration, and a concomitant improvement of functional recovery; however, only a small percentage of axons regrow, highlighting the need for the removal of other impediments to regeneration for a more complete therapeutic solution (Simonen M., et al. (2003) Neuron 38: 201; Zheng B., et al. (2003) Neuron 38: 213).

The main component of glial scarring is astrocytic gliosis, whereby normally quiescent astrocytes show a vigorous response to injury. (Stichel CC, et al. (1998) Cell Tissue Res 294: 1). They become hypertrophic, proliferative, upregulate expression of glial fibrillary acidic protein (GFAP), and form a dense network of glial processes both at and extending from the lesion site. At the same time, the astrocytes secrete a variety of cytokines and produce cell adhesion and extracellular matrix molecules, some of which are inhibitory to regeneration (e.g., chondroitin sulfate proteoglycan (CSPG) and collagen IV). Blocking the deposition of said astrocytic products can promote axonal regeneration is promoted. (Stitchel C C, et al.).

As most spinal cord injury-related attempted therapeutics to date have centered on overcoming either myelin inhibitors or components of the glial scar, agents aimed at inhibiting Eph receptors (e.g., the compounds of the invention) are representative of a new strategy to promote nerve regeneration.

Eph Receptors and Ephrins

The Eph receptor tyrosine kinase subfamily appears to be the largest subfamily of transmembrane receptor tyrosine kinases, and with its ligands, the ephrins, is responsible for governing proper cell migration and positioning during neural development, presumably through modulating intercellular repulsion (Pasquale, E. (1997) Curr. Opin. Cell Biol. 9:608-615)(Orioli and Klein (1997) Trends in Genetics 13:354-359). The Eph family is responsible for the formation of the corticospinal tract and anterior commissure. (Kullander K., et al. (2001a) Neuron 29: 73; Henkemeyer M, et al. (1996) Cell 86: 35).

Eph receptors are closely related, and actively signal when bound to their ephrin ligands (their effects are mediated by cell-to-cell contacts), with which they are capable of both forward and bi-directional signaling. (Murai, K. K., et al. (2003) J Cell Sci. 116(14): 2823).

These receptors are characterized by 3 functional domains: an intracellular tyrosine kinase catalytic domain, a single membrane spanning domain, and an extracellular ligand binding domain.

Binding of a ligand ephrin by a Eph receptor induces phosphorylation on tyrosine residues, which establishes binding sites for signaling proteins containing SH2 domains and activates an array of signaling pathways. The ephrins are thought to activate Eph receptors by clustering them and inducing autophosphorylation, while soluble monomeric ephrins are thought to inhibit Eph receptor activation. (Davis et al. (1994) Science 266: 816).

The sixteen known Eph receptors are divided into two subgroups (EphA and EphB) based on sequence homology. EphA receptors preferentially bind the glycosylphosphatidylinositol (GPI)-linked ephrin-A ligands, while EphB preferentially receptors bind the transmembrane ephin-B ligands. However, the ephrin ligands are rather promiscuous, and tend to lack selectivity in their activation of Eph receptors. (Murai, K. et al. (2003) Molecular and Cellular Neuroscience 24: 1000). For instance, EphA4 can bind (and is therefore activated by) ligand ephrins B2 and B3, in addition to members of the ephrin A ligand family.

Eph receptor family members and their ephrin ligands are of interest as targets for therapy for the treatment of neurological disorders and injuries, including as targets for the promotion of axon regeneration, based on findings in the literature. For instance, because Eph-ephrin signaling appears to regulate axon guidance through contact repulsion, inducing the collapse of neuronal growth cones (Wahl S., et al. (2000) J Cell Biol 149: 263; Kullander et al.), and members of this family are upregulated in the adult after neural injury (Moreno-Flores M T, et al. (1999) Neuroscience 91: 193; Willson C A, et al. (2002) Cell Transplant 11: 229), the aberrant expression or absence of Eph receptors could prove pivotal in determining the outcome of injury in the adult CNS.

EphA4

EphA4 is a receptor tyrosine kinase from the EphA family which has important functions in the developing and adult nervous system. Along with its known expression pattern during neural development (Mori, T., et al. (1995) Brain Res Mol Brain Res 29:325; Ohta, K., et al. (1996) Mechanisms of Development 54:59; Soans, C., et al. (1994) Oncogene 9:3353), EphA4 is expressed in brain regions that show extensive synaptic remodeling (Murai, K., et al. (2003) Nature Neurosci 6:153). In the adult, EphA4 is enriched in the hippocampus and cortex, two brain structures critical for learning and memory. The receptor is also enriched in migrating neural crest cells, growing axonal projections, and mature brain structures that show extensive plasticity. (Murai, et al.).

A recent study implicates EphA4 in two critical aspects of spinal cord injury, axonal inhibition and astrocytic gliosis. (Goldshmit, Y., et al. (2004) J Neurosci. 24(45): 10064). Goldshmit compared neural regeneration after spinal cord hemisection in wild-type and EphA4−/− mice, and discovered an overall functional improvement in the latter, characterized by a lack of astrocytic gliosis and regeneration of ipsilateral axons. Regarding the mechanisms through which improvements were seen, the experiments (as well as literature) demonstrates three roles for Eph receptors in axonal regeneration:

The first, as demonstrated by in vitro assays, is the direct inhibition of neurite outgrowth mediated by EphA4 on the astrocytes binding to a receptor-ligand on the axon. Such an action of EphA4 may provide a mechanism for the inhibition of neurite outgrowth on astrocytes observed in the presence of IFN, which Goldshmit has shown upregulates EphA4 expression. (Fok-Seang J., et al. (1998) Eur J Neurosci 10: 2400). These results suggest that EphA4 is yet another directly inhibitory molecule produced during astrocytic gliosis, in addition to other inhibitory components, such as extracellular matrix and myelin-derived molecules.

The second, and lesser-observed, mechanism may be by activation of EphA4 on the regenerating axons, similar to on El6 cortical neurons. However, EphA4 was found to be highly expressed only on astrocytes and motor neurons, and present at low levels on descending axons in lesioned adult spinal cord.

The third mechanism by which EphA4 exerts an inhibitory effect involves its vital role in activating astrocytes, leading to gliosis and the formation of a glial scar. Such activation appears to be dependent on responsiveness to cytokine stimulation and may be dependent on Rho activation. This cytokine-induced response may be attributable to the upregulation of EphA4 receptor expression on the astrocytes, allowing enhanced ligand binding and receptor activation. It is also possible that the cytokine-induced astrocyte proliferation and hypertrophy may be caused by transactivation of EphA4, as has been shown for FGF2- and PDGF-induced phosphorylation of EphrinB molecules (Chong et al., (2000) Mol Cell Biol 20: 724), leading to Rho activation and cytoskeletal rearrangement. The difference in glial activation seems to be astrocyte specific as there was no apparent difference in macrophage-microglial activation. Ephs and Ephrins have been reported to play a role in interactions between astrocytes and meningeal fibroblasts, excluding fibroblasts from the glial scar. (Bundesen L Q, et al. (2003) J Neurosci 23: 7789).

Process of Manufacture

Compounds of formula I are prepared analogously to methods that, for other compounds, are in principle known in the art, preferably by reacting a boronic acid derivative of the formula II,

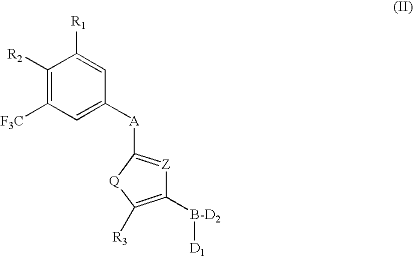

wherein D₁ and D₂ are hydroxy or substituted hydroxy, or together with the binding boron atom and two binding oxygen atoms form a ring of the formula IIA,

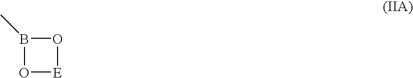

wherein E is alkylene, substituted alkylene, unsubstituted or substituted cycloalkylene, unsubstituted or substituted bicycloalkylene or unsubstituted or substituted tricycloalkylene,

with a coupling partner of the formula III,

R₄-L (III)

wherein R₄ is as defined above or below for a compound of the formula I and L is a leaving group;

and, if desired, transforming a compound of formula I into a different compound of formula I, transforming a salt of an obtainable compound of formula I into the free compound or a different salt, and/or transforming an obtainable free compound of formula I into a salt thereof.

The reaction preferably takes place under customary conditions e.g. for the Suzuki-Miyaura cross-coupling (see e.g. Miyaura et al., Chem. Rev. 95, 2457 (1995)), in the presence of an appropriate (preferably water-free=absolute) solvent, for example an ether, such as ethylene glykol dimethyl ether or dioxane, a haydrocarbon, such as hexanes or toluene, or an alcohol, such as ethanol, or a mixture of any two or more thereof, in the presence of a catalyst, especially a noble metal complex catalyst, for example an iridium, a rhodium or preferably a palladium catalyst, such as tetrakis(triphenylphosphin)-palladium (Pd(PPh₃)₄) (which may also be formed in situ, e.g. from a palladium salt, such as palladium acetate, and the complex ligand, e.g. triphenylphosphin), preferably in the presence of a base, e.g. an acid addition salt of a metal, such as an alkali metal salt of an inorganic acid, e.g. a (e.g. sodium or potassium) phosphate or carbonate, or of a carbonic acid, e.g. a (e.g. sodium or potassium) lower alkanoate, such as acetate, at preferably elevated temperatures, e.g. between 25° C. and the reflux temperature, e.g. between 75 and 95° C. The reaction preferably takes place under an inert gas, such as nitrogen or argon.

If D₁ and D₂ each are substituted hydroxy, then substituted hydroxy is preferably alkyloxy, especially lower alkyloxy, aryloxy, especially phenyloxy with unsubstituted or substituted phenyl as defined above, or cycloalkyloxy wherein cycloalkyl is preferably C₃-C₈-cycloalkyl, such as cyclopentyl or cyclohexyl.

If (as is preferred) D₁ and D₂ together with the binding boron atom and oxygen atoms form a ring or the formula IIA shown above, then E preferably carries the two oxygen atoms bound to the boron atom on two different carbon atoms that are spatially nearby or neighboring carbon atoms, e.g. in vicinal (“1,2-”) or in “1,3”-position (relatively to each other).

Alkylene is preferably an unbranched C₂-C₁₂-, preferably C₂-C₇alkylene moiety, e.g. ethylene, or propylene, in a broader aspect butylene, pentylene or hexylene, bound via two different carbon atoms as just described, preferably vicinal or in “1,3”-position. Substituted alkylene (which is preferred) is preferably an unbranched lower alkylene moiety as defined above which is substituted or unsubstituted by one or more, especially up to four, substituents preferably independently selected from lower alkyl, such as methyl or ethyl, e.g. in 1-methylethylene, 1,2-dimethylethylene, (preferably) 2,2-dimethylpropylene (neopentylene) or (especially preferred) 1,1,2,2-tetramethylethylene, or in a broader sense of the invention hydroxy, e.g. in 2-hydroxy-propylene, or hydroxy-lower alkyl, such as hydroxymethyl, e.g. in 1-hydroxymethyl-ethylene.

Unsubstituted or substituted cycloalkylene is preferably C₃-C₁₂-, more preferably C₃-C₈-cycloalkylene bound via two different carbon atoms as described for W, preferably vicinal or in “1,3”-position, such as cyclohexylene or cyclopentylene. Unsubstituted or substituted bicycloalkylene is preferably C₅-C₁₂-bicycloalkylene bound via two different carbon atoms as described for E, preferably vicinal or in “1,3”-position. An example is pinanylene (2,3-(2,6,6-trimethyl-bicyclo[3. 1. I ]heptane). Unsubstituted or substituted tricycloalkylene is preferably C₈-C₁₂-tricycloalkylene bound via two different carbon atoms as described for E, preferably vicinal or in “1,3”-position.

Unsubstituted or substituted cycloalkylene, unsubstituted or substituted bicycloalkylene or unsubstituted or substituted tricycloalkylene may be unsubstituted or substituted by one or more, especially up to three substituents independently selected from lower alkyl, such as methyl or ethyl, hydroxy, hydroxy-lower alkyl, such as methoxy, or mono- or oligosaccharidyl bound via an oxygen atom (“oligosaccharidyl” preferably comprising up to five saccharidyl moieties).

A leaving group L in a compound of the formula III is preferably halo, especially iodo, bromo (preferred) or chloro, or perfluoroalkylsulfonyloxy (e.g. —O—SO₂—(C_{f}F_{2f+1}), wherein f=1, 2 or 4).

In principle, manufacture of a compound of the formula I is alternatively also possible employing a compound of the formula II with a leaving group L instead of the group of the formula IIA given above and, as reaction partner, a compound of the formula III bearing a group of the formula IIA given above instead of the leaving group L. The reaction conditions then are analogous to those described for the reaction of the compounds of formula II and III given above.

Optional Reactions and Conversions

Compounds of the formula I may be converted into different compounds of the formula I. For example, lower alkoxycarbonyl substituents may be converted into carboxyl by saponification, nitro substituents may be hydrogenated to amino.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent. Internal salts of compounds of formula I containing acid and basic salt-forming groups, e.g. a free carboxy group and a free amino group, may be formed, e.g. by the neutralisation of salts, such as acid addition salts, to the iso-electric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Intermediates and final products can be worked up and/or purified according to standard methods, e.g. using chromatographic methods, distribution methods, (re-) crystallization, and the like.

Starting Materials

The starting materials can, for example, preferably be prepared as follows:

A boronic acid derivative of the formula II is preferably prepared by reacting a compound of the formula IV,

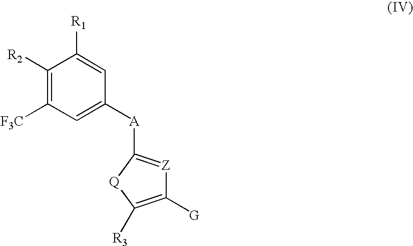

wherein R₁, R₂, R₃, A, Q and Z are as defined above for a compound of the formula I and G is a leaving group, especially as defined above for the leaving group L in a compound of the formula III, with a diboron compound of the formula VA or VB,

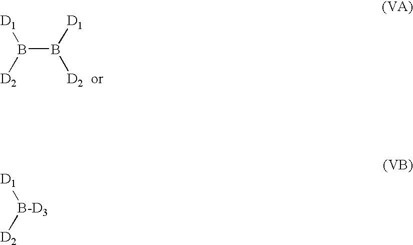

wherein D₁ and D₂ are as defined above for a compound of the formula II and D₃ is substituted hydroxy as defined above under formula II, under customary reaction conditions, that is in the presence of a in the presence of an appropriate (preferably water-free=absolute) solvent, for example an ether, such as ethylene glykol dimethyl ether, tetrahydrofurane or dioxane, a hydrocarbon, e.g. hexanes, or an alcohol, such as ethanol, or a mixture of any two or more thereof, in the presence of a noble metal complex catalyst, such as an iridium, rhodium or preferably palladium, e.g. preferably 1,1′-bis(diphenylphosphino)ferrocene-dichloro palladium (Pd(dppf)Cl₂), complex catalyst, and preferably in the presence of a base, e.g. an acid addition salt of a metal, such as an alkali metal salt of an inorganic acid, e.g. a (e.g. sodium or potassium) carbonate, or of a carbonic acid, e.g. a (e.g. sodium or potassium) lower alkanoate, such as acetate, at preferred temperatures e.g. between 20° C. and the reflux temperature, e.g. between 75 and the reflux temperature of the reaction mixture. The reaction preferably takes place under an inert gas, such as nitrogen or argon. Alternatively, the compound of the formula IV can first be lithiated, e.g. with n-butyllithium, and the resulting lithiated product then reacted with the compound of the formula VB under customary reaction conditions.

A starting material of the formula IV wherein R₁, R₂, R₃, Q and Z are as defined above or below for a compound of the formula I and G is a leaving group and A is —C(═O)—NH— (with the —NH— bound to the ring comprising Q and Z in formula I) is preferably manufactured by reacting a reactive derivative of a carbonic acid of the formula VI,

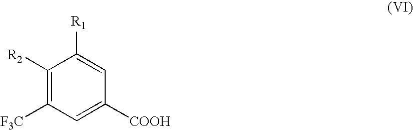

wherein R₁ and R₂ are as defined for a compound of the formula I, with an amino base of the formula VII,

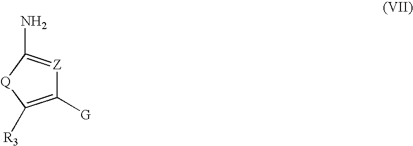

wherein Q, Z and R₃ and are as defined for a compound of the formula I and G is a leaving group as defined under formula IV, in an appropriate solvent, e.g. a nitrile, such as acetonitrile, preferably at a temperature from 0 to 50° C., e.g. from 20 to 40° C., preferably in the presence of a base, e.g. a tertiary nitrogen base, such as a tri-lower alkylamine, e.g. triethylamine. The active derivative is either converted in situ into a reactive derivative, e.g. by dissolving the compounds of formulae IV and V in a suitable solvent, for example N,N-dimethylformamide, N,N-dimethylacetamide, N-methyl-2-pyrrolidone, methylene chloride, or a mixture of two or more such solvents, and by the addition of a suitable base, for example triethylamine, diisopropylethylamine (DIEA) or N-methylmorpholine and a suitable coupling agent that forms a preferred reactive derivative of the carbonic acid of formula III in situ, for example dicyclohexylcarbodiimide/1-hydroxybenzotriazole (DCC/HOBT); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); or 1-(3-dimethylaminopropyl)-3-ethylcarbodiimide hydrochloride (EDC). For review of other possible coupling agents, see e.g. Klauser; Bodansky, Synthesis 1972, 453-463. The reaction mixture is preferably stirred at a temperature of between approximately −20 and 50° C., especially between 0° C. and room temperature, to yield a compound of formula IV. Alternatively, the carbonic acid of the formula VI is used in the form of a reactive derivative, e.g. as the carbonic acid halide, such as chloride, as an anhydride with a carbonic acid, e.g. with a C₁-C₇-alkanoic acid, as an active ester, or in the form of an alkali metal salt, e.g. a sodium, lithium or potassium salt. In both cases, the reaction can preferably be carried out under an inert gas, e.g. nitrogen or argon.

A starting material of the formula IV wherein R₁, R₂, R₃, Q and Z are as defined above or below for a compound of the formula I and G is a leaving group and A is —NH—C(═O)— (with the —C(═O)— bound to the ring comprising Q and Z in formula I) can be synthesized from a reactive derivative (formed in situ or directly present, see the analogous reaction conditions using reactive derivatives of carbonic acids of the formula VI above) of a carbonic acid of the formula VIII,

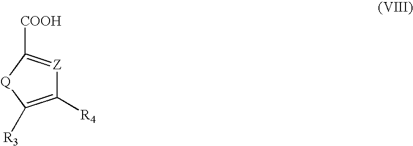

wherein R₃, Q and Z are as defined for a compound of the formula I and G is a leaving group as defined under formula IV, by reaction with an amino compound of the formula IX,

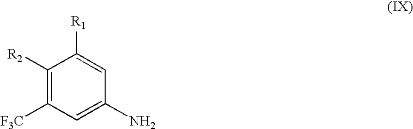

wherein R₁ and R₂ are as defined for a compound of the formula I, where the reaction conditions being used are analogous to those described herein for reaction of a compound of the formula VI and VII.

A compound of the formula III wherein L is a perfluoroalkanesulfonyloxy leaving group can be prepared, for example, by reacting a corresponding compound wherein instead of L a hydroxy group is present with a corresponding perfluoroalkanesulfonic anhydride, e.g. in an appropriate solvent, such as a halogenated hydrocarbon, e.g. dichloromethylene, in the presence of a (preferably tertiary nitrogen) base, such as a tri-lower alkylamine, e.g. triethylamine, a preferred temperatures from −10° C. to 50° C., e.g. from 0° C. to 25° C. A compound of the formula III wherein L is halo can, for example, be prepared by reacting a corresponding precursor compound wherein instead of L hydrogen is present, with a halogenating agent, e.g. N-bromosuccinimide in concentrated sulfuric acid/trifluoro acetic acid at preferred temperatures between 0 and 40° C., e.g. at room temperature.

Other starting materials, e.g. of the formula V, VI, VII, VIII and IX, are known, can be obtained in analogy to methods that are known in the art and/or are commercially available, especially by or in analogy to methods given in the examples.

General Process Conditions

The following applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this text, only a readily removable group that is not a constituent of the particular desired end product of formula I is designated a “protecting group”, unless the context indicates otherwise. The protection of functional groups by such protecting groups, the protecting groups themselves, and the reactions appropriate for their removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about —100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

The compounds, which term is in each case including the free compounds and/or their salts where salt-forming groups are present, may also be obtained in the form of hydrates, or their crystals may, for example, include the solvent used for crystallization, forming solvates. Different crystalline forms may be present.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula I described as being preferred. Special preference is given to reaction conditions that are identical or analogous to those mentioned in the Examples.

### Preferred Embodiments According to the Invention

In the following preferred embodiments, any one or more general expressions can be replaced by the corresponding more specific definitions provided above and below, thus yielding stronger preferred embodiments of the invention.

A preferred embodiment of the invention relates to a compound of the formula I wherein Q is —CH═CH— and R₁, R₂, R₃, R_{4,} R₅, A and Z are as defined for a compound of the formula I, or a (preferably pharmaceutically acceptable) salt thereof; or its use.

Another preferred embodiment of the invention relates to a compound of the formula I wherein A is —C(═O)—NH— (with the —NH— bound to the ring comprising Q and Z in formula I) and R₁, R₂, R₃, R_{4,} R₅, Q and Z are as defined for a compound of the formula I, or a (preferably pharmaceutically acceptable) salt thereof; or its use.

Another preferred embodiment relates to a compound of the formula I wherein one of R₁ and R₂ is hydrogen and the other is hydrogen or a moiety selected from the group consisting of

for R₂:

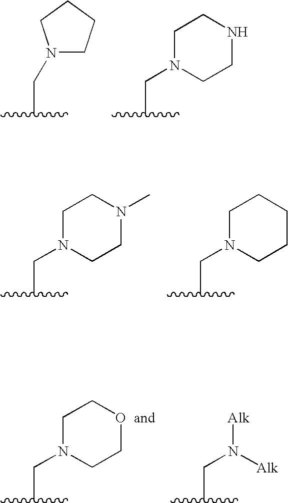

for R₁:

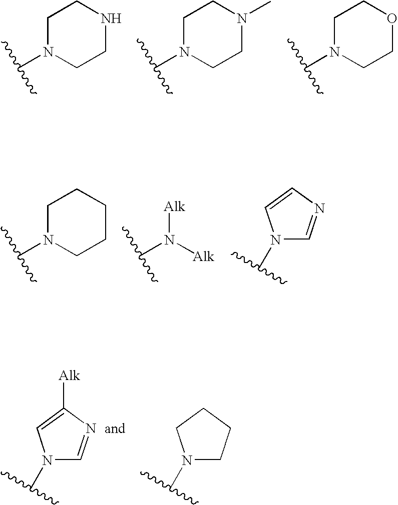

wherein “Alk” is alkyl, preferably lower alkyl, more preferably methyl or ethyl; and R₃, R_{4,} R₅, A, Q and Z are as defined above or below for a compound of the formula I, or a (preferably pharmaceutically acceptable) salt thereof.

The invention relates more preferably to a compound of the formula I, wherein

each of R₁ and R₂ is hydrogen;

R₃ is C₁-C₇-alkyl, especially methyl;

R₄ is bicyclic heterocyclyl selected from the group consisting of

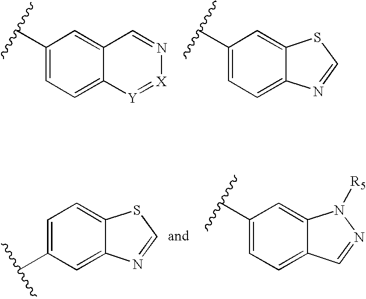

wherein

X is CH, N or C—NH₂;

Y is CH or N;

with the proviso that not both of X and Y are N simultaneously;

and R₅ is hydrogen, C₁-C₇-alkyl or phenyl;

(wherein R₄ is preferably

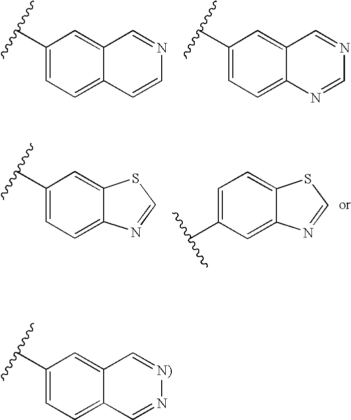

A is —C(═O)—NH— (with the —NH— bound to the ring comprising Q and Z in formula I) or —NH—C(═O)— (with the —C(═O)— bound to the ring comprising Q and Z in formula I);

Z is CH; and

Q is —CH═CH—;

or a (preferably pharmaceutically acceptable) salt thereof where one or more salt-forming groups are present.

A preferred embodiment of the invention relates to the use (as defined above) of a compound of the formula I, or a pharmaceutically acceptable salt thereof; wherein Q is S and R₁, R₂, R₃, R_{4,} R₅, A and Z are as defined above or below for a compound of formula I.

Preferred is also the use (as defined above) of a compound of the formula I, or a pharmaceutically acceptable salt thereof, wherein A is NH—C(═O) (with the —C(═O)— bound to the ring comprising Q and Z in formula I) and R₁, R₂, R₃, R_{4,} R₅, Q and Z are as defined above or below for a compound of the formula I.

Especially preferred is the use of a compound of formula (I), or a pharmaceutically acceptable salt thereof, in the manufacture of a pharmaceutical preparation for the treatment of an Eph receptor-related (e.g., neurological) injury and disorder. Also preferred is a compound of the formula (I), or a pharmaceutically acceptable salt thereof, as shown above for use in the treatment of an Eph receptor-related (e.g., neurological) injury and disorder.

Pharmaceutical Compositions

The invention relates also to the use of pharmaceutical compositions comprising a compound of formula (I) in the therapeutic (in a broader aspect of the invention also prophylactic) treatment of an Eph receptor-related (e.g., neurological) injury and disorder.

The pharmacologically acceptable compounds of the present invention may be used, for example, for the preparation of pharmaceutical compositions that comprise an effective amount of a compound of the formula (I), or a pharmaceutically acceptable salt thereof, as active ingredient together or in admixture with a significant amount of one or more inorganic or organic, solid or liquid, pharmaceutically acceptable carriers.

The invention relates also to a pharmaceutical composition that is suitable for administration to a warm-blooded animal, especially a human (or to cells or cell lines derived from a warm-blooded animal, especially a human, e.g. lymphocytes), for the treatment or, in a broader aspect of the invention, prevention of (=prophylaxis against) a disease that responds to inhibition of kinase activity, comprising an amount of a compound of formula (I) or a pharmaceutically acceptable salt thereof, which is effective for said inhibition, especially the in, together with at least one pharmaceutically acceptable carrier.

The pharmaceutical compositions according to the invention are those for enteral, such as nasal, rectal or oral, or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to a method of treatment for a disease that responds to inhibition of a kinase; which comprises administering an (against the mentioned disease) prophylactically or especially therapeutically effective amount of a compound of formula (I) according to the invention, especially to a warm-blooded animal, for example a human, that, on account of one of the mentioned diseases, requires such treatment.

The dose of a compound of the formula (I) or a pharmaceutically acceptable salt thereof to be administered to warm-blooded animals, for example humans of approximately 70 kg body weight, is preferably from approximately 3 mg to approximately 10 g, more preferably from approximately 10 mg to approximately 1.5 g, most preferably from about 100 mg to about 1000 mg/person/day, divided preferably into 1-3 single doses which may, for example, be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragees, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22, especially from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C8 to C12, Hüls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragee cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragee coatings or the capsule casings, for example for identification purposes or to indicate different doses of active ingredient.

Combinations

The compounds of the invention may also be used to advantage in combination with other agents known to overcome process outgrowth inhibition such as Rho kinase inhibitors; inhibitors of classical PKC isoforms; blocking antibodies against NogoA or the Nogo receptor; Chondroitinase ABC or other reagents that cleave the GAG side chains off proteoglycans; and agents that increase intrinsic growth capacity of neurons (e.g., cAMP and bcl-2).

By way of a non-exclusive example, the compounds of the invention may be used in combinatorial therapy with an agent capable of blocking myelin inhibitors Nogo, myelin-associated glycoprotein (MAG), or oligodendrocyte-myelin glycoprotein OMgp.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula (I), can be prepared and administered as described in the art such as in the documents cited above.

The following examples are merely illustrative and not meant to limit the scope of the present claims in any manner.

### Examples

Examples 1-12

### Syntheses

The following examples employ the abbreviations listed herein, and unless otherwise listed, the following conditions: where no temperatures are given, the reaction takes place at ambient (room) temperature; and ratios of solvents, e.g., in eluents or solvent mixtures, are given in volume by volume (v/v).

Ratios of solvents, e.g., in eluents or solvent mixtures, are given in volume by volume (v/v) or in volume percent. Temperatures are measured in degrees Celsius. Unless otherwise indicated, the reactions take place at RT. The R_{f} values which indicate the ratio of the distance moved by each substance to the distance moved by the eluent front are determined on silica gel thin-layer plates (Merck, Darmstadt, Germany) by thin-layer chromatography using the respective named solvent systems.

The analytical HPLC conditions where HPLC is mentioned are as follows:

Column: (70×4.0 mm) HPLC column CC 70/4 Nucleosil 100-3 C18 (3 μm mean particle size, with silica gel covalently derivatized with octadecylsilanes, Macherey & Nagel, Düren, Germany). Detection by UV absorption at 215 nm. The retention times (t_{R}) are given in minutes. Flow rate: 1 ml/min.

Gradient: 20%→100% a) in b) for 5 min+1 min 100% a). a): Acetonitrile+0.1% TFA; b): water +0.1% TFA.

Other HPLC Conditions:

HPLC(GRAD3):

Column: (250×4.6 mm) packed with reversed-phase material C18-Nucleosil (5 μm mean particle size, with silica gel covalently derivatized with octadecylsilanes, Macherey & Nagel, Düren, Germany). Detection by UV absorption at 215 nm. The retention times (t_{R}) are given in minutes. Flow rate: 1 ml/min.

Gradient: 5%→40% a) in b) for 7.5 min+7 min 40% a). a): Acetonitrile+0.1% TFA; b): water+0.1% TFA.

The short forms and abbreviations used have the following definitions:

conc.: concentrated; DMF: N,N-dimethylformamide; MS-ES: mass spectroscopy (electron spray); h: hour(s); Me: methyl; min: minute(s); mL: milliliter(s); m.p.: melting point; RT: room temperature; TFA: trifluoroacetic acid; THF: tetrahydrofuran (distilled over Na/benzophenone); TLC: thin-layer chromatography; t_{R}: retention times

### Example 1

### N-(3-Isoquinolin-7-yl-4-methyl-phenyl)-3-trifluoromethyl-benzamide (Compound 1)

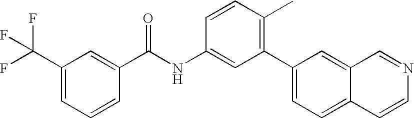

To a solution of N-[4-methyl-3-(4,4,5,5-tetramethyl-[1,3,2]dioxaborolan-2-yl)-phenyl]-3-trifluoromethyl-benzamide (1.74 g, 4.3 mmol) and trifluoro-methanesulfonic acid isoquinolin-7-yl ester (1.081 g, 3.9 mmol) in 28 mL of dry dioxane, 1.23 g (5.79 mmol) potassium phosphate are added and the solution is degassed by bubbling a slow stream of nitrogen through the suspension during 15 min. After the addition of 0.232 g (0.33 mmol) tetrakis(triphenylphosphin)palladium the mixture is heated for 10 h to 90° C. The same amount of catalyst and potassium phosphate is added again, and the mixture is then stirred for 17 h at 90° C. The reaction mixture is cooled, filtered through Hyflo Super Cele® (Fluka, Buchs, Switzerland) and the residue washed with dioxane. The combined dioxane solutions are evaporated and the brown residue is purified by chromatography using a 120 g silica gel column on a Combi-Flash Companion™ (Isco Inc.) apparatus. A gradient of tert-butyl-methylether/hexane 1:1 to 4:1 is used. Pure fractions are pooled and evaporated to give the title compound as a pink foam; R_{f}(tert-butyl-methylether)=0.32; HPLC t_{R}=3.24 min; MS-ES+: (M+H)+=407.

### Step 1.1: N-[4-Methyl-3-(4,4,5,5-tetramethyl-[1,3,2]dioxaborolan-2-yl)-phenyl]-3-trifluoromethyl-benzamide

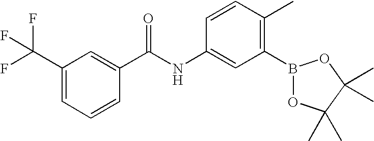

Nitrogen is bubbled through a mixture of 5.0 g (14 mmol) N-(3-bromo-4-methyl-phenyl)-3-trifluoromethyl-benzamide and 3.42 g (34.5 mmol) potassium acetate in 50 mL of THF for about 20 minutes. After the addition of 4.06 mg (16 mmol) bis-(pinacolato)-diboron, 6 mol-% of 1,1′-bis(diphenylphospino)ferrocene-palladium dichloride (700 mg, 0.8 mmol) is added and the resulting mixture heated under reflux for 18 h. The reaction mixture is then cooled to RT and diluted with ethyl acetate. After washing the mixture with conc. Sodium chloride solution, the ethyl acetate phase is dried with sodium sulphate and evaporated. The crude product is purified by flash chromatography using dichloromethane as solvent. The title compound is obtained as a colorless solid; m.p. 148-152° C.; R_{f}(dichloromethane)=0.36; HPLC t_{R}=4.82 min; MS-ES+: (M+H)+=406.

### Step 1.2: N-(3-Bromo-4-methyl-phenyl)-3-trifluoromethyl-benzamide

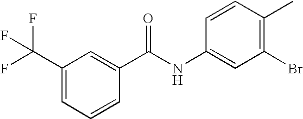

A solution of 5.8 mL (39 mmol) 3-trifluoromethyl-benzoyl chloride in 80 mL acetonitrile is treated drop-wise and at RT with 12.2 mL (78 mmol) triethylamine, followed by 7.8 g (42.9 mmol) 3-bromo-4-methyl-aniline. During the slow addition of the 3-trifluoromethyl-aniline, the temperature rises to about 30° C. The mixture is stirred at room temperature for 10 h and then cooled to 0° C. Water is added (100 mL) and the resulting precipitate filtered off, washed with water and dried. The solid is suspended in hexane stirred for a few min, filtered and dried again to give the title compound as a colorless solid; m.p. 153-155° C.; HPLC t_{R}=4.54 min.

### Step 1.3: Trifluoro-methanesulfonic acid isoguinolin-7-yl ester

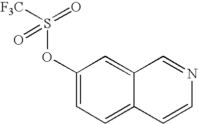

A solution of 5.8 g (0.04 mol) 7-hydroxyquinoline and 6.68 mL (0.048 mol) triethylamine in 100 mL of dichloromethane is cooled in an ice bath and treated dropwise over 30 min with 7.26 mL (0.044 mol) trifluoro-sulfonic acid anhydride. After complete addition, the cooling bath is removed and the dark mixture stirred for 1.5 h at RT. The reaction mixture is poured into 100 mL of ice-water and the bi-phasic mixture filtered through Hyflo Super Cel® (filtering aid based on diatomaceous earth; obtainable from Fluka, Buchs, Switzerland). The organic layer is separated and washed with 50 mL 10% citric acid, 50 mL of brine, dried with sodium sulphate and evaporated to leave a brown resin. This is purified by flash chromatography using dichloromethane/ethyl acetate 100:2.5 to 100:5. Pure fractions are pooled and evaporated to give an orange oil. HPLC t_{R}=2.35 min; R_{f}(tert-butyl-methylether)=0.38; MS-ES+: (M+H)+=278.

### Example 2

### N-(4-Methyl-3-quinazolin-6-yl-phenyl)-3-trifluoromethyl-benzamide (Compound 2)

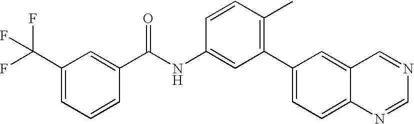

A mixture of N-[4-methyl-3-(4,4,5,5-tetramethyl-[1,3,2]dioxaborolan-2-yl)-phenyl]-3-trifluoromethyl-benzamide (0.456 g, 1.125 mmol) and 6-bromo-quinazoline (0.157 g, 0.75 mmol) in 3 mL of toluene and 0.375 mL of ethanol is treated with 0.75 mL of a 2 molar solution of sodium carbonate and the resulting mixture is degassed by bubbling nitrogen through the mixture for 5 min. After the addition of palladium acetate (0.0075 g, 0.034 mmol) and triphenylphosphine (0.0293 g, 0.117 mmol), the mixture is stirred at 90° C. for 2 h. The same amount of palladium acetate and triphenylphosphine is added again and the mixture stirred for 6 h at 90° C. The reaction mixture is cooled and added to 10 mL ethyl acetate and 4 mL of water. The bi-phasic mixture is filtered through Hyflo Super Cel® (Fluka, Buchs, Switzerland), the organic layer separated, dried with sodium sulphate and evaporated to leave a brown resin. The crude product is purified by chromatography using a 40 g silica gel column on a Combi-Flash Companion™ (Isco Inc.) apparatus. A gradient of dichloromethane/methanol 100:1 to 100:15 is used. Enriched fractions are re-chromatographed on the same system using a 40 g silica gel column and tert-butyl-methylether as solvent. Pure fractions are pooled and evaporated to give the title compound as a tan foam; R_{f}(dichloromethane/ethanol 9:1)=0.56; HPLC t_{R}=3.23 min; MS-ES+: (M+H)+=408.

### Step 2.1: 6-Bromo-quinazoline

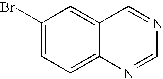

Trifluoroacetic acid (10 mL) is placed in a reaction vessel equipped with a thermometer and a mechanical stirrer. At 20° C., quinazoline (2.6 g, 0.020 mol) is added, followed by 3.4 mL of 96% sulphuric acid. N-Bromosuccinimide (4.8 g, 0.027 mol) is then added in 5 portions allowing 30 min in between the additions. After complete addition, the yellow mixture is stirred for 17 h at RT. The trifluoroacetic acid is removed on a rotary evaporator (rotavap) and the residue poured onto 20 g of crashed ice. The pH of the mixture is adjusted to 8-9 by the addition of 30% sodium hydroxide solution. The resulting suspension is diluted with 40 mL of ethyl acetate and filtered. The organic layer is separated and the aqueous phase extracted with 20 mL of ethyl acetate. The combined ethyl acetate extracts are dried with sodium sulphate and evaporated. Flash-chromatography of the residue using ethyl acetate/hexane 1:3 to 1:2 gives the title compound as colorless crystals. m.p. 155-156° C.; HPLC t_{R}=1.29 min; R_{f}(ethyl acetate/hexane 3:2)=0.36; MS-ES+: (M+H)+=210.9.

### Example 3

### 3-Isoquinolin-7-yl-4-methyl-N-(3-trifluoromethyl-phenyl)-benzamide (Compound 3)

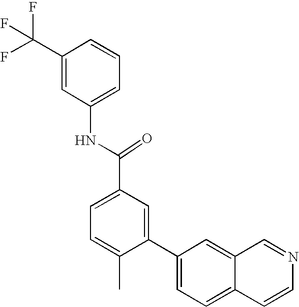

Using 4-methyl-3-(4,4,5,5-tetramethyl-[1,3,2]dioxaborolan-2-yl)-N-(3-trifluoromethyl-phenyl)-benzamide as different starting material, the same procedure as described in example 1 is used, except that no second addition of catalyst is required. The title compound is obtained as colorless solid; m.p. 189-191° C.; HPLC t_{R}=3.30 min; R_{f}(ethyl acetate/dichloromethane 1:4)=0.21; MS-ES+: (M+H)+=407.

### Step 3.1: 4-Methyl-3-(4,4,5,5-tetramethyl-[1,3,2]dioxaborolan-2-yl)-N-(3-trifluoromethyl-phenyl)-benzamide

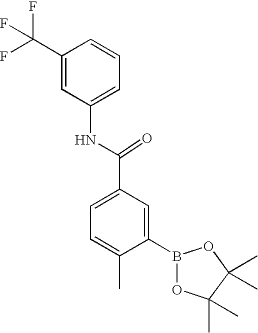

The same procedure is used as described in example 1, step 1.1 but starting with 3-bromo-4-methyl-N-(3-trifluoromethyl-phenyl)-benzamide. Reaction time is 8 h. The title compound is obtained as a tan solid; m.p. 157-159° C.; R_{f}(dichloromethane)=0.36; HPLC t_{R}=4.93 min; MS-ES+: (M+H)+=406.

### Step 3.2: 3-Bromo-4-methyl-N-(3-trifluoromethyl-phenyl)-benzamide

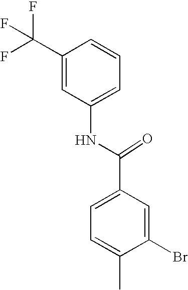

A solution of 14 g (60 mmol) 3-bromo-4-methyl-benzoyl chloride in 120 mL acetonitrile is treated drop-wise and at RT with 12.6 g (120 mmol) triethylamine, followed by 8.3 mL (66 mmol) 3-trifluoromethyl-aniline. During the slow addition of the 3-trifluoromethyl-aniline the temperature rises to about 35° C. The mixture is stirred at room temperature for 5 h and then diluted with ethyl acetate. The resulting mixture is washed sequentially with saturated sodium bicarbonate solution, 1 N hydrochloric acid and brine and then dried with sodium sulphate. Evaporation of the solvent leaves a brown oil which is crystallized from ether/petrol-ether to give the title compound as a colorless solid; m.p. 157-158° C.; HPLC t_{R}=4.63 min; R_{f}(dichloromethane)=0.75.

### Example 4

### 4-Methyl-3-quinazolin-6-yl-N-(3-trifluoromethyl-phenyl)-benzamide (Compound 4)

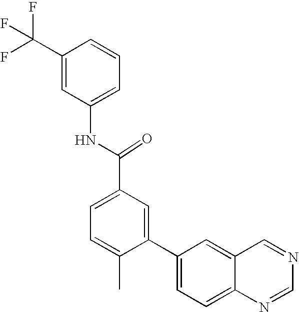

Using the title compound of example 3.1 as differing starting material, the same procedure as described in example 2 is used, except that no second addition of catalyst is required. The title compound is obtained as a colorless foam; HPLC t_{R}=3.31 min; R_{f}(tert.-butyl-methylether)=0.21; MS-ES+: (M+H)+=408.

### Example 5

### N-(3-Benzothiazol-6-yl-4-methyl-phenyl)-3-trifluoromethyl-benzamide (Compound 5)

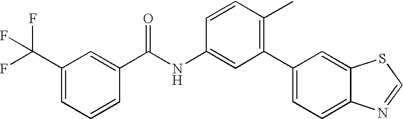

Using 6-bromo-benzothiazol as the differing starting material, the same procedure as described in example 2 is used, except that no second addition of catalyst is required. Reaction time 2 h, purification by flash chromatography. The title compound is obtained as a colorless solid; m.p. 94-96° C.; HPLC t_{R}=4.58 min; R_{f}(dichlorome-thane/ethanol 98:2)=0.3; MS-ES+: (M+H)+=413.

### Example 6

### 3-Benzothiazol-6-yl-4-methyl-N-(3-trifluoromethyl-phenyl)-benzamide (Compound 6)

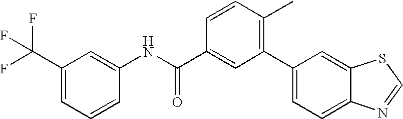

Using 6-bromo-benzothiazol and the title compound of example 3.1 as starting materials, the same procedure as described in example 2 is used, except that no second addition of catalyst is required. Reaction time 3 h. The title compound is obtained as a colorless solid; m.p. 102-104° C.; HPLC t_{R}=4.66 min; R_{f}(dichloromethane/ethanol 98:2)=0.3; MS-ES+: (M+H)+=413.

### Example 7

### N-(4-Methyl-3-phthalazin-6-yl-phenyl)-3-trifluoromethyl-benzamide (Compound 7)

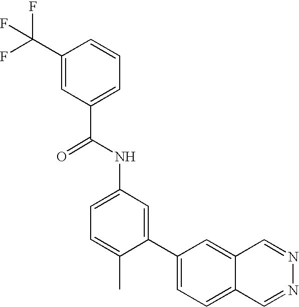

The same procedure as described in example 2 is used, except that no second addition of catalyst is required. Reaction time 3 h. The title compound is obtained as a colorless solid; m.p. 205-206° C.; HPLC t_{R}=3.34 min; MS-ES⁺: (M+H)⁺=408.

The starting material is prepared as follows:

### Step 7.1: 6-Bromo-phthalazine

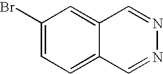

A solution of 1.0 g (4.7 mmol) 4-bromo-benzene-1,2-dicarbaldehyde in 4 mL of ethanol and 4 ml of dichloromethane is added dropwise over 40 min at 0° C. and under nitrogen to a solution of hydrazine hydrate (0.684 mL, 14.1 mmol) in 4.7 mL of ethanol. The resulting suspension is stirred 1 h at 0° C. and then the solvent is evaporated. The crystalline material is stirred with 20 mL of toluene and the solvent is evaporated again. This procedure is repeated with dichloromerthane. At the end the product is dried at 60° C. under vacuum for 8 h to give the title compound as colorless crystals: m.p. 140-143° C., HPLC t_{R}=1.49 min; ME-ES⁺: (M+H)⁺=210.9.

### Step 7.2: 4-Bromo-benzene-1,2-dicarbaldehyde

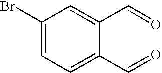

The title compound is synthesized by Swern oxidation of (4-bromo-2-hydroxymethyl-phenyl)-methanol following the procedure by O. Farooq, Synthesis 10, 1035-1037 (1994) and obtained as slightly yellow crystals: m.p. 97-100° C., MS-ES⁺: (M+H)⁺=210.9+212.9.

### Step 7.3: 3-(4-Bromo-2-hydroxymethyl-phenyl)methanol

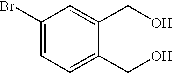

To a solution of3 g (12.2 mmol) 4-bromo-phthalic acid in 24 mL of 1,2-dimethoxyethane, at 0° C. 1.394 g (36.8 mmol) of sodium borohydride are added in 10 portions. After stirring for 15 min, a solution of 4.61 mL (36.5 mmol) boron trifluoride etherate in 8 mL of 1,2-dimethoxyethane is added within 10 min. After stirring for 10 min at 0° C., the mixture is allowed to warm up to RT and stirring is continued for 2 h. The reaction mixture is then slowly added onto 40 g of crushed ice and the aqueous mixture is evaporated with ethyl acetate. The combined ethyl acetate axtracts are washed with water and brine, dried with sodium sulfate and evaporated. The residual yellow oil (crude material) is purified by chromatography using a 120 g silica gel column on a Combi-Flash Companion (Isco Inc.) chromatography apparatus. A gradient of dichloromethane/ethyl acetate 0−>50% ethyl acetate is used. The title compound is obtained as an oil which crystallizes on standing: m.p. 79-81° C., HPLC t_{R}=1.94 min, MS-ES⁺: (M+H)⁺=214+216.

### Example 8

### 4-Methyl-3-phthalazin-6-yl-N-(3-trifluoromethyl-phenyl)-benzamide (Compound 8)

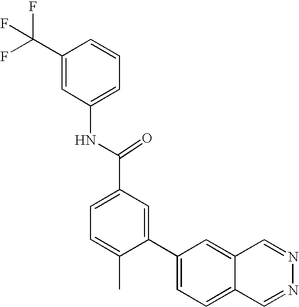

The same procedure as described in Example 7 is used. Title compound: m.p. 270-272° C.; HPLC t_{R}=3.43 min; R_{f}(dichloromethane/ethanol)=0.32; MS-ES⁺ (M+H)⁺=408.

### Example 9

### N-(3-Benzothiazol-5-yl-4-methyl-phenyl)-3-trifluoromethyl-benzamide (Compound 9)

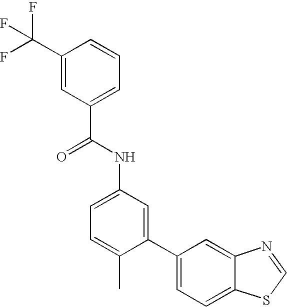

The same procedure as described in Example 2 is used starting with 5-bromo-benzothiazole. Reaction time total 4 h. The title compound is obtained as a colorless solid. M.p. 90-93° C., HPLC t_{R}=4.54 min; R_{f}(dichloromethane(ethanol) 0.30; MS-ES⁺: (M+H)⁺=413.

The starting material is prepared as follows:

### Step 9.1: 5-Bromo-benzothiazole

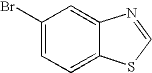

4-Amino-benztothiazole (3.0 g, 0.02 mol) in 18 mL of a 35% hydrobromic acid solution is diazotized at 0° C. by slow addition of a solution of 1.19 g (0,0195 mmol) sodium nitrite in 11 mL of water. After stirring for 1 h at 0° C. the brown solution is added dropwise to a dark solution of 3.3 g (0.023 mol) CuBr in 45 mL of a 35% hydrobromic acid solution at 0° C. The reaction mixture is stirred 0.5 h at 0° C., 2 h at RT and then 2h at 90° C. The mixture is cooled to RT and pored into 20 g of crushed ice. Concentrated ammonia is added to the mixture to make it alkaline and then it is extracted with ethyl acetate. The organic layers are combined, washed with brine, dried with sodium sulfate and evaporated. The residue is purified by flash chromatography on silica gel using dichloromethane/petrol ether as eluent. The title compound is obtained as a solid: m.p. 104-106° C., HPLC t_{R}=3.44 min; R_{f}(dochloromethane/petrol ether)=0.30.

### Step 9.2: 5-Amino-benzothiazole

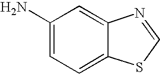

Purified 5-nitro-benzothiazole (7.2 g, 0.04 mol, see WO 98/23612, example 7A), dissolved in 160 mL of methanol and 160 mL of THF, is hydrogenated in the presence of 1.6 g Pd/C (10%; Engelhard 4505). The catalyst is filtered off, the filtrate concentrated and the residual oil purified by flash chromatography on silica gel using dichloromethanol/methanol 97:3 as eluent. The title compound is obtained as a colorless solid: m.p. 76-78° C., HPLC t_{R}=0.76 min; MS-ES⁺: (M+H)⁺=151; R_{f}(dichloromethane/methanol 97:3)=0.76.

### Example 10

### 3-Benzothiazol-5-yl-4-methyl-N-(3-trifluoromethylphenyl)benzamide (Compound 10)

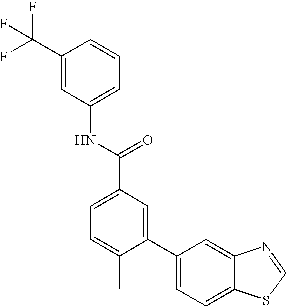

The same procedure as described in Example 9 is used. Title compound: m.p. 200-202° C., HPLC t_{R}=4.62 min; R_{f}(dichloromethane/ethanol 98:2)=0.30; MS-ES⁺: (M+H)⁺=413.

### Example 11

### Soft Capsules

5000 soft gelatin capsules, each comprising as active ingredient 0.05 g of one of the compounds of formula I mentioned in any one of the preceding Examples, are prepared as follows:

Composition:

Active ingredient: 250 g

Lauroglycol: 2 litres

Preparation process: The pulverized active ingredient is suspended in Lauroglykol® (propylene glycol laurate, Gattefossé´ S.A., Saint Priest, France) and ground in a wet pulverizer to produce a particle size of about 1 to 3 μm. 0.419 g portions of the mixture are then introduced into soft gelatin capsules using a capsule-filling machine.

### Example 12

### Tablets comprising compounds of the formula I

Tablets, comprising, as active ingredient, 100 mg of any one of the compounds of formula I of Examples 1 to 10 are prepared with the following composition, following standard procedures:

Composition:

Active Ingredient: 100 mg; crystalline lactose: 240 mg; Avicel: 80 mg; PVPPXL: 20 mg; Aerosil: 2 mg; magnesium stearate: 5 mg, TOTALLING: 447 mg

Manufacture: The active ingredient is mixed with the carrier materials and compressed by means of a tabletting machine (Korsch EKO, Stempeldurchmesser 10 mm).

Avicel® is microcrystalline cellulose (FMC, Philadelphia, USA). PVPPXL is polyvinylpolypyrrolidone, cross-linked (BASF, Germany). Aerosil® is silcium dioxide (Degussa, Germany).

### Example 13

### EphA4 Mode and Mechanism of Action

In order to distinguish between the forward and bi-directional signaling that ephrins are capable of in the context of axon regeneration, lentiviral expression vectors for wild type and kinase dead EphA4 are generated and overexpressed in purified astrocytes. Cortical neurons are plated on the two astrocytic populations and neurite outgrowth assayed and compared. Biological peptides that have been demonstrated to block the interaction of EphA4 with relevant ligands, consequently inhibiting receptor activation (Murai, K. K., et al., (2003) Mol Cell Neurosci 24(4): p. 1000), are tested for their EphA4 inhibitory activity in the astrocyte/cortical neuron culture system. Identification of the neuronal ligand/ephrin mediating EphA4 inhibition is achieved by systematically blocking candidate ephrin expression in neurons using RNA interference to knock down ephrins or by using dominant negative ephrin constructs and subsequently plating them on wild type astrocytes. These experiments collectively clarify the mode of EphA4 activation.

To illustrate intracellular events triggered by EphA4 activation, cytokine induced activation of astrocytes are used to explore the precise signaling pathways activated. Cultured astrocytes are treated with inflammatory cytokines (which have been shown to be involved in activating astrocytes) LIF or IFN in the presence or absence of EphA4 blocking peptides, and the cells are lysed and analyzed by Western Blots for the activation of major signaling pathways (MAPK, P13K, JNK, STAT, RhoA) using appropriate phospho-antibodies. The signaling involved in neurite outgrowth inhibition by EphA4 is assessed by culturing cortical neurons on astrocytes or on CNS myelin or spinal cord extracts in the presence or absence of commercially available pharmacological inhibitors of the major signaling pathways and also the EphA4 inhibitory peptides.

### Example 14

### Autophosphorylation and Ligand-Dependent Phosphorylation Assays

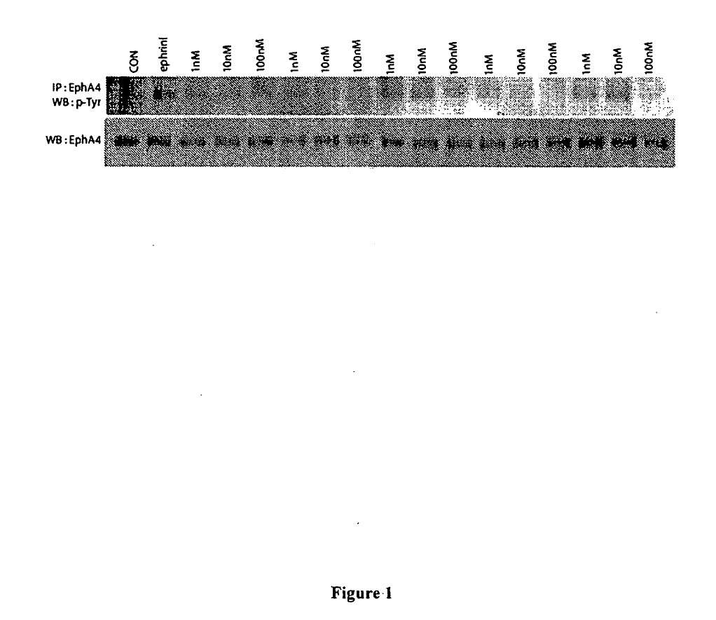

Primary astrocyte cultures are established from neonatal mouse cortex and purified so as to get about 95-98% pure astrocyte cultures. For detecting autophosphorylation the cells are incubated in the presence or absence of pharmacological inhibitors and then directly lysed and subjected to immunoprecipitation and Western analysis. For ligand dependent phosphorylation (as seen in FIG. 1), the cultures are then serum starved for 36 hours to reduce basal receptor phosphorylation and then stimulated for varying lengths of time with a soluble form of the cognate ligand in the presence or absence of candidate kinase inhibitors or blocking peptides, which are added at various concentrations. Cells are lysed, and the lysates subjected to EphA4 immunoprecipitation and subsequently analyzed on Westerns for level of receptor phosphorylation using a phospho-tyrosine antibody.

### Example 15

### In vitro Assay for Neurite Outgrowth/Axon Regeneration

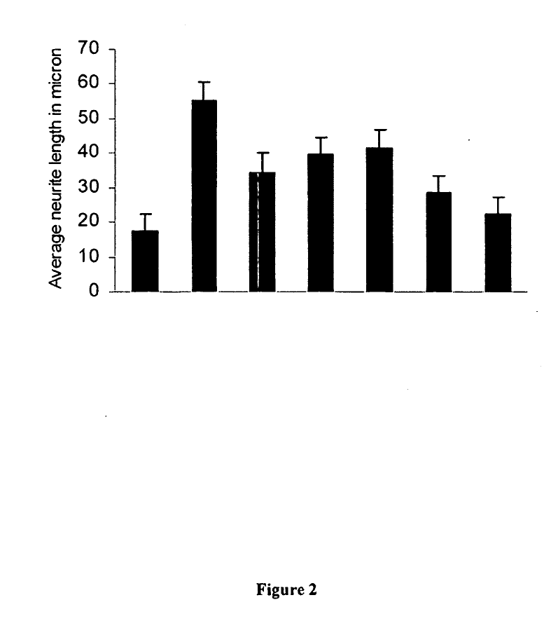

This assay is used to assess neurite outgrowth inhibition of embryonic cortical neurons by Eph receptors expressed on astrocytes or neurite outgrowth inhibition of post-natal cortical neurons by ephrin ligand present in myelin. Post natal(P3) cortical neurons are plated onto immobilized CNS myelin in 4-well chamber slide or 96-well plates. Pharmacological inhibitors, are added to the medium and the length of longest neurite from each neuron is measured under each condition and compared to average neurite length on myelin in the absence of any pharmacological agents. FIG. 2 depicts the quantitation of neurite outgrowth effects observed with Compound 3 and other compounds (all tested at 100 nM concentration) in cortical cultures plated on CNS myelin.

### Example 16

### In vitro Assay for Astrogliosis—Astrocyte Scratch Wound

Assay Astrocytes are prepared from the cerebral cortex of neonatal C57BL/6 mice(P1-P2). Cells are maintained in Dulbecco's modified Eagle's medium with 10% FBS. 4-7 weeks old astrocytes are plated to confluence in 2 well chamber slides coated with poly-D-lysine for the scratch wound assay and serum starved. 48 hrs after serum starvation, the monolayer of astrocytes is scratched with sterile 200 μl tips and washed twice with PBS to get rid of cell debris. Conditioned medium (±cytokines) is added to the wounded astrocytes. The microscopic images of the scratch is captured at a magnification of 10× right after scratch and considered as time point 0.24 hrs, 48 hrs or 72 hrs after scratch, the same region of scratch is imaged and fixed with methanol containing 1 μg/ml of DAPI to monitor migration and proliferation of astrocytes.

### Example 17

### Proof of mechanism

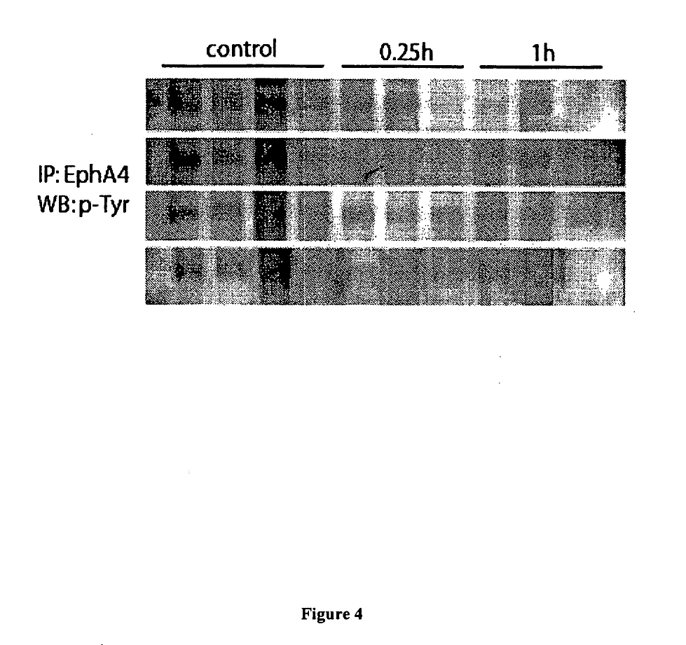

The experiment demonstrates that the compounds of the invention cross the blood brain barrier and effectively block phosphorylation of EphA4 receptor in vivo. Male NMRI mice were injected with relevant compounds at a dose of 10 mg/kg body weight and were sacrificed either 25 minutes or 1 hour following the dosing (0.25 h or 1 h shown in FIG. 4). The brains were removed and one half of each brain was weighed and homogenized in appropriate volume of lysis buffer for 30 seconds (10 seconds pulse and 10 seconds off—3 times). The homogenate was spun at 12,000 g for 30 minutes. Protein amounts were estimated for supernatants (using BCA) and equal amounts of protein for each condition were subjected to EphA4 immunoprecipitation followed by a phospho-tyrosine western blot. Four control animals were used and three experimental animals per time point were used for each of the compounds tested.

### Example 18

### High Throughput screening (HTS)

High throughput screens can be developed to look for selective and specific pharmacological inhibitors of EphA4 activity. Such compounds, as with the compounds of the invention, include kinase inhibitors or binding antagonists that block EphA4 interaction with its ligand and/or specifically block EphA4 kinase activation. Such compounds, as with the compounds of the invention, can serve as inhibitors that efficiently block EphA4 activity in the context of gliosis and axon regeneration

### Example 19

### In vivo Target Validation in a Mouse SCI Model

Existing EphA4 inhibitory peptides/hits from the HTS described herein, e.g., the compounds of the invention can be used for in vivo spinal cord injury (SCI) experiments to determine their efficacy in promoting axon regeneration. Mice are divided into three groups: unlesioned; lesioned with vehicle infusion; and lesioned with drug/peptide infusion. Animals of the lesioned groups undergo spinal hemisection surgery. Drug or vehicle (e.g., containing one of the compounds of the invention) is administered intrathecally via an osmotic pump, and an anterograde tracer is used to track anatomical regeneration of lesioned axons. Appropriate behavioral and electrophysiological assays can be performed to assess functional recovery of sensory and motor functions.

In addition to the SCI model experiments described above, EphA4 inhibitory agents, e.g., the compounds of the invention, can also be tested while Nogo signaling is compromised, to see if this results in a synergistic effect leading to improved functional recovery.

What is claimed is:

1. A method of treating an Eph receptor-related injury or disorder comprising administering a compound of formula (I) to a warm-blooded animal, especially a human, in need of such treatment: 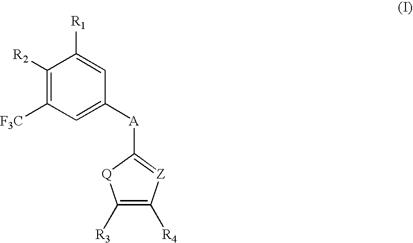
wherein
R₁ is hydrogen or —N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;
R₂ is hydrogen or —CH₂—N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;
with the proviso that at least one of R₁ and R₂ is hydrogen;
R₃ is halo or C₁-C₇-alkyl;
R₄ is bicyclic heterocyclyl selected from the group consisting of 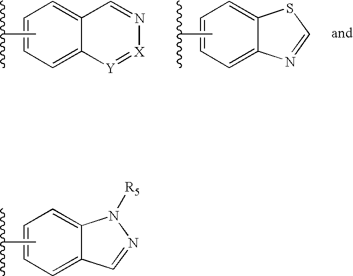
wherein
X is CH, N or C—NH₂;
Y is CH or N;
with the proviso that not both of X and Y are N simultaneously; and
R₅ is hydrogen, C₁-C₇-alkyl or unsubstituted or substituted phenyl;
A is —C(═O)—NH— with the —NH— bound to the ring comprising Q and Z in formula I or —NH—C(═O)— with the —C(═O)— bound to the ring comprising Q and Z in formula I;
Z is CH or N; and
Q is —S— or —CH═CH—;
or a salt thereof where one or more salt-forming groups are present.

2. The method according to claim 1, further comprising administering a compound of formula I, wherein Q is —CH═CH— and R₁, R₂, R₃, R_{4,} R₅, A and Z are as defined in claim 1, or a—preferably pharmaceutically acceptable—salt thereof.

3. The method according to claim 1, further comprising administering a compound of formula I, wherein A is —C(═O)—NH— with the —NH— bound to the ring comprising Q and Z in formula I and R₁, R₂, R₃, R₄, R₅, Q and Z are as defined in claim 1, or a—preferably pharmaceutically acceptable—salt thereof.

4. The method according to claim 1, further comprising administering a compound of formula I, wherein one of R₁ and R₂ is hydrogen and the other is hydrogen or a moiety selected from the group consisting of
for R₂: 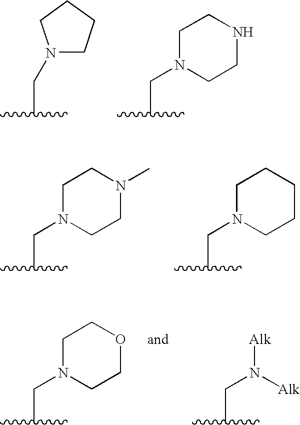
for R₁: 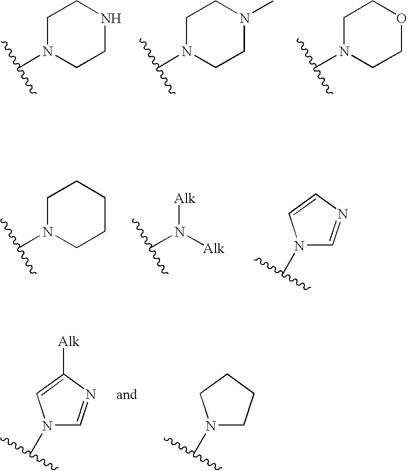
wherein “Alk” is alkyl, preferably lower alkyl, more preferably methyl or ethyl; and R₃, R_{4,} R₅, A, Q and Z are as defined in claim 1, or a—preferably pharmaceutically acceptable—salt thereof.

5. The method according to claim 1, further comprising administering a compound of formula I, wherein
each of R₁ and R₂ is hydrogen;
R₃ is C₁-C₇-alkyl, especially methyl;
R₄ is bicyclic heterocyclyl selected from the group consisting of 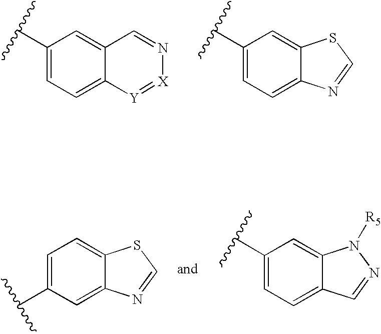
wherein
X is CH, N or C—NH₂;
Y is CH or N;
with the proviso that not both of X and Y are N simultaneously;
and R₅ is hydrogen, C₁-C₇-alkyl or phenyl;
(wherein R₄ is preferably 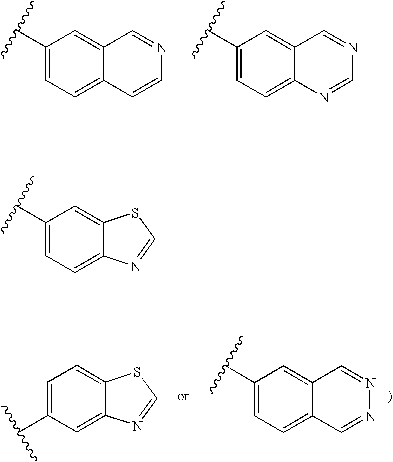
A is —C(═O)—NH— (with the —NH— bound to the ring comprising Q and Z in formula I) or —NH—C(═O)— (with the —C(═O)— bound to the ring comprising Q and Z in formula I);
Z is CH; and
Q is —CH═CH—;
or a—preferably pharmaceutically acceptable—salt thereof where one or more salt-forming groups are present.

6. The method according to claim 1, further comprising administering a compound of formula I, selected from the group consisting of
N-(3-isoquinolin-7-yl-4-methyl-phenyl)-3-trifluoromethyl-benzamide,
N-(4-methyl-3-quinazolin-6-yl-phenyl)-3-trifluoromethyl-benzamide,
3-isoquinolin-7-yl-4-methyl-N-(3-trifluoromethyl-phenyl)-benzamide,
4-methyl-3-quinazolin-6-yl-N-(3-trifluoromethyl-phenyl)-benzamide,
N-(3-benzothiazol-6-yl-4-methyl-phenyl)-3-trifluoromethyl-benzamide,
3-benzothiazol-6-yl-4-methyl-N-(3-trifluoromethyl-phenyl)-benzamide,
N-(4-methyl-3-phthalazin-6-yl-phenyl)-3-trifluoromethyl-benzamide,
4-methyl-3-phthalazin-6-yl-N-(3-trifluoromethyl-phenyl)-benzamide,
N-(3-benzothiazol-5-yl-4-methyl-phenyl)-3-trifluoromethyl-benzamide, and
3-benzothiazol-5-yl-4-methyl-N-(3-trifluoromethylphenyl)benzamide
or a pharmaceutically acceptable salt thereof where a salt-forming group is present.

7. The method according to claim 1, further comprising a compound selected from the group of Compounds 1-10.

8. The method according to claim 1, wherein the disease to be treated is a neurodegenerative disease.

9. The method according to claim 1, wherein the Eph receptor-related injury or disorder is quadriplegia, hemiplegia, and paraplegia.

10. The method of claim 9, wherein the quadriplegia, hemiplegia, and paraplegia is caused by injury or trauma.

11. The method of claim 9, wherein the quadriplegia, hemiplegia, and paraplegia is caused by hereditary illness.

12. A method according to claim 1, wherein the injury to be treated is or results from a spinal cord injury.

13. A method according to claim 1, wherein the injury to be treated results from a cerebral infarct such as in stroke.

14. A method of treating an Eph receptor-related injury or disorder comprising administering a pharmaceutical composition comprising a compound of formula (I), or a pharmaceutically acceptable salt thereof, and a pharmaceutically acceptable carrier, to a warm-blooded animal, especially a human, in need of such treatment: 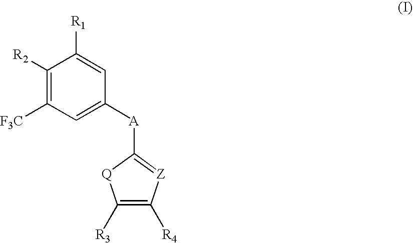
wherein
R₁ is hydrogen or —N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;
R₂ is hydrogen or —CH₂—N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;
with the proviso that at least one of R₁ and R₂ is hydrogen;
R₃ is halo or C₁-C₇-alkyl;
R₄ is bicyclic heterocyclyl selected from the group consisting of 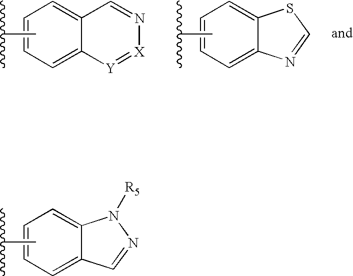
wherein
X is CH, N or C—NH₂;
Y is CH or N;
with the proviso that not both of X and Y are N simultaneously; and
R₅ is hydrogen, C₁-C₇-alkyl or unsubstituted or substituted phenyl;
A is —C(═O)—NH— with the —NH— bound to the ring comprising Q and Z in formula I or —NH—C(═O)— with the —C(═O)— bound to the ring comprising Q and Z in formula I;
Z is CH or N; and
Q is —S— or —CH═CH—;
or a salt thereof where one or more salt-forming groups are present.

15. The method according to claim 14, further comprising administering a pharmaceutical composition comprising a compound of formula I, wherein Q is —CH═CH— and R₁, R₂, R₃, R_{4,} R₅, A and Z are as defined in claim 14, or a pharmaceutically acceptable salt thereof.

16. The method according to claim 14, further comprising administering a pharmaceutical composition comprising a compound of formula I, wherein A is —C(═O)—NH— with the —NH— bound to the ring comprising Q and Z in formula I and R₁, R₂, R₃, R_{4,} R₅, Q and Z are as defined in claim 14, or a pharmaceutically acceptable salt thereof.

17. The method according to claim 14, further comprising administering a pharmaceutical composition comprising a compound of formula I, wherein one of R₁ and R₂ is hydrogen and the other is hydrogen or a moiety selected from the group consisting of
for R₂: 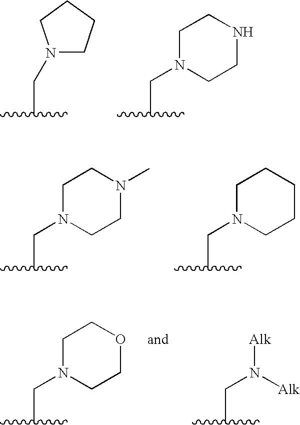
for R₁: 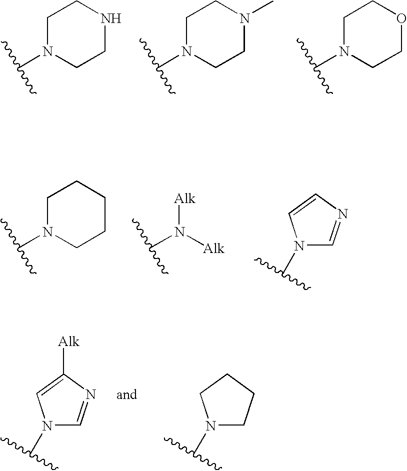
wherein “Alk” is alkyl, preferably lower alkyl, more preferably methyl or ethyl; and R₃, R_{4,} R₅, A, Q and Z are as defined in claim 1, or a pharmaceutically acceptable salt thereof.

18. The method according to claim 14, further comprising administering a compound of formula I, wherein
each of R₁ and R₂ is hydrogen;
R₃ is C₁-C₇-alkyl, especially methyl;
R₄ is bicyclic heterocyclyl selected from the group consisting of 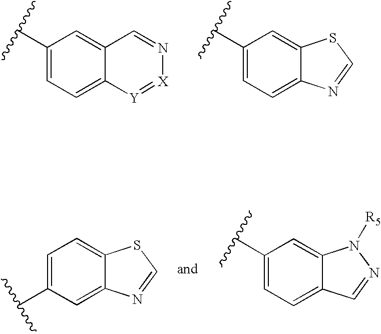
wherein
X is CH, N or C—NH₂;
Y is CH or N;
with the proviso that not both of X and Y are N simultaneously;
and R₅ is hydrogen, C₁-C₇-alkyl or phenyl;
(wherein R₄ is preferably 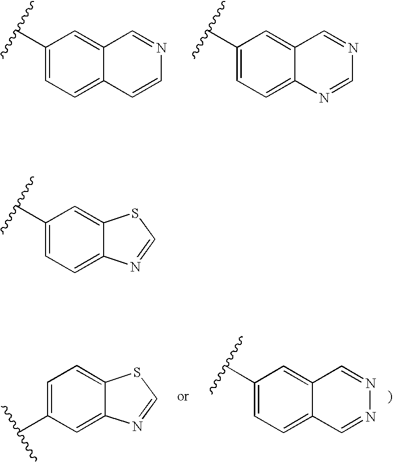
A is —C(═O)—NH— (with the —NH— bound to the ring comprising Q and Z in formula I) or —NH—C(═O)— (with the —C(═O)— bound to the ring comprising Q and Z in formula I);
Z is CH; and
Q is —CH═CH—;
or a pharmaceutically acceptable salt thereof where one or more salt-forming groups are present.

19. The method according to claim 14, further comprising administering a compound of formula I, selected from the group consisting of
N-(3-isoquinolin-7-yl-4-methyl-phenyl)-3-trifluoromethyl-benzamide,
N-(4-methyl-3-quinazolin-6-yl-phenyl)-3-trifluoromethyl-benzamide,
3-isoquinolin-7-yl-4-methyl-N-(3-trifluoromethyl-phenyl)-benzamide,
4-methyl-3-quinazolin-6-yl-N-(3-trifluoromethyl-phenyl)-benzamide,
N-(3-benzothiazol-6-yl -4-methyl-phenyl)-3-trifluoromethyl-benzamide,
3-benzothiazol-6-yl-4-methyl —N-(3-trifluoromethyl-phenyl)-benzamide,
N-(4-methyl-3-phthalazin-6-yl-phenyl)-3-trifluoromethyl-benzamide,
4-methyl-3-phthalazin-6-yl-N-(3-trifluoromethyl-phenyl)-benzamide,
N-(3-benzothiazol-5-yl-4-methyl-phenyl)-3-trifluoromethyl-benzamide, and
3-benzothiazol-5-yl-4-methyl-N-(3-trifluoromethylphenyl)benzamide or a pharmaceutically acceptable salt thereof where a salt-forming group is present.

20. The method according to claim 14, further comprising a compound selected from the group of Compounds 1-10.

21. The method according to claim 20, wherein the disease to be treated is a neurodegenerative disease.

22. The method according to claim 20, wherein the Eph receptor-related injury or disorder is quadriplegia, hemiplegia, and paraplegia.

23. The method of claim 22, wherein the quadriplegia, hemiplegia, and paraplegia is caused by injury or trauma.

24. The method of claim 22, wherein the quadriplegia, hemiplegia, and paraplegia is caused by hereditary illness.

25. A method according to claim 20, wherein the injury to be treated is or results from a spinal cord injury.

26. A method according to claim 20, wherein the injury to be treated results from a cerebral infarct such as in stroke.

27. A method of stimulating neural regeneration, or reversing neuronal degeneration, or both, comprising administering a compound of formula (I) to a warm-blooded animal, especially a human: 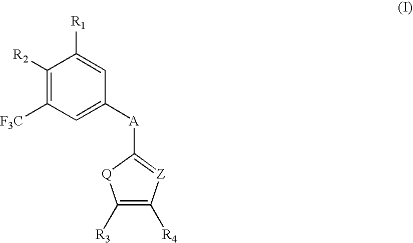
wherein
R₁ is hydrogen or —N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;
R₂ is hydrogen or —CH₂—N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;
with the proviso that at least one of R₁ and R₂ is hydrogen;
R₃ is halo or C₁-C₇-alkyl;
R₄ is bicyclic heterocyclyl selected from the group consisting of 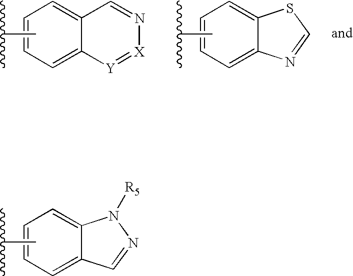
wherein
X is CH, N or C—NH₂;
Y is CH or N;
with the proviso that not both of X and Y are N simultaneously; and
R₅ is hydrogen, C₁-C₇-alkyl or unsubstituted or substituted phenyl;
A is —C(═O)—NH— with the —NH— bound to the ring comprising Q and Z in formula I or —NH—C(═O)— with the —C(═O)— bound to the ring comprising Q and Z in formula I;
Z is CH or N; and
Q is —S— or —CH═CH—;
or a salt thereof where one or more salt-forming groups are present.

28. The method of claim 27, wherein the warm-blooded animal has suffered a neuronal injury.

29. The method of claim 27, wherein the warm-blooded animal suffers from a neurological disorder.

30. The method of claim 27, wherein the warm-blooded animal suffers from quadriplegia, hemiplegia, and paraplegia caused by hereditary illness.

31. The method of claim 27, wherein the warm-blooded animal suffers from a spinal cord injury.

32. The method of claim 27, wherein the warm-blooded animal has experienced a cerebral infarct such as in stroke.

33. The method of claim 1, wherein the compound of formula (I) is combined in a combination therapy with an agent capable of blocking myelin inhibitors Nogo, myelin-associated glycoprotein (MAG), or oligodendrocyte-myelin glycoprotein OMgp.
